(19) United States
(12) Patent Application Publication
(10) Pub. No.: US 2020/0281943 A1
(43) Pub. Date: Sep. 10, 2020

(54) NEUROACTIVE STEROIDS AND THEIR METHODS OF USE

(71) Applicant: Sage Therapeutics, Inc., Cambridge, MA (US)

(72) Inventors: Ethan Hoffmann, Sherborn, MA (US); George Nomikos, Cambridge, MA (US); Handan Gunduz-Bruce, Lexington, MA (US); Stephen Jay Kanes, Brynlawn, PA (US)

(21) Appl. No.: 16/644,680

(22) PCT Filed: Sep. 7, 2018
(86) PCT No.: PCT/US2018/050012

§ 371 Date: Mar. 5, 2020

Related U.S. Application Data
(60) Provisional application No. 62/595,998, filed on Dec. 7, 2017.
(60) Provisional application No. 62/555,404, filed on Sep. 7, 2017.

(51) Int. Cl. A61K 31/58

(52) U.S. Cl. CPC A61K 31/58; A61K 9/20

(57) ABSTRACT

This disclosure relates to Compound (I), pharmaceutically acceptable salts, or pharmaceutically acceptable compositions thereof, for the treatment of CNS related disorders, e.g., tremor, e.g., essential tremor; depression; and anxiety disorder, and methods for improving the effectiveness of the administration of Compound 1 for treating said CNS related disorders. The disclosure also relates to methods of increasing the bioavailability of a Compound 1 or pharmaceutically acceptable salts, or pharmaceutically acceptable compositions thereof.

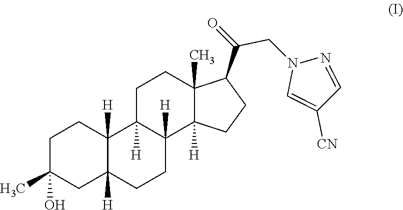

### CROSS-REFERENCE TO RELATED APPLICATIONS

This application claims priority to U.S. Ser. No. 62/555,404 filed Sep. 7, 2017 and U.S. Ser. No. 62/595,998 filed Dec. 7, 2017, the contents of each of which are incorporated herein by reference in their entirety.

### BACKGROUND OF THE INVENTION

GABA, γ-aminobutyric acid, has a profound influence on overall brain excitability because up to 40% of the neurons in the brain utilize GABA as a neurotransmitter. GABA interacts with its recognition site on the GRC (GABA receptor complex) to facilitate the flow of chloride ions down an electrochemical gradient of the GRC into the cell. An intracellular increase in the levels of this anion causes hyperpolarization of the transmembrane potential, rendering the neuron less susceptible to excitatory inputs (i.e., reduced neuron excitability). In other words, the higher the chloride ion concentration in the neuron, the lower the brain excitability (the level of arousal). It is well-documented that the GRC is responsible for the mediation of anxiety, seizure activity, and sedation. Thus, GABA and drugs that act like GABA (e.g., the therapeutically useful barbiturates and benzodiazepines (BZs), such as Valium®) produce their therapeutically useful effects by interacting with specific regulatory sites on the GRC.

Accumulated evidence has indicated that the GRC contains a distinct site for neuroactive steroids (Lan, N. C. et al., Neuwchem. Res. 16:347-356 (1991)). Neuroactive steroids can occur endogenously. The most potent endogenous neuroactive steroids are 3α-hydroxy-5-reduced pregnan-20-one and 3cc-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232: 1004-1007 (1986); Harrison, N. L. et al., J Pharmacol. Exp. Ther. 241:346-353 (1987)).

Compound 1, a neuroactive steroid described herein, has been shown to be a positive allosteric modulator of GABA_{A} receptors that targets synaptic and extrasynaptic GABA_{A} receptors. As a positive allosteric modulator of GABA_{A} receptors, Compound 1 serves as a therapeutic agent to treat CNS related disorders, e.g., tremor, e.g., essential tremor; depression, e.g., postpartum depression; and anxiety disorder. Although many routes may be used for administering therapeutic agents, the oral route is preferred due to its convenience. However, many therapeutically active agents experience low bioavailability after oral administration due to a myriad of issues, including poor absorption or susceptibility to first pass metabolism. Furthermore, solid dosage forms, e.g. tablets and capsules, are preferable over other dosage form as they are taken orally by patients, which is a convenient and safe way of drug administration and they are more stable compared to liquids (physical and chemical stability). Yet, solid dosage forms administered orally suffer from low bioavailability. In order for Compound 1 to be administered orally as a therapeutic agent for CNS related disorders, especially in a solid dosage form, an acceptable bioavailability must be achieved.

### SUMMARY OF THE INVENTION

The disclosure provides methods of treating a CNS related disorder by administering a therapeutically effective amount of Compound 1 or 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile, which has the formula

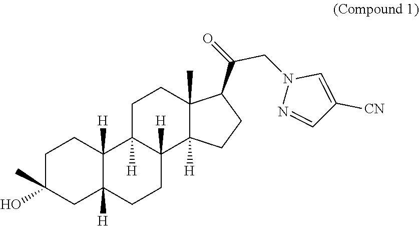

in a subject in need thereof. Compound 1 is also known as the compound SAGE-217.

In some embodiments, Compound 1 is administered without food or substantially contemporaneously with food. In some embodiments, Compound 1 is administered substantially contemporaneously with food.

In some embodiments, the method comprises a method of treating a CNS related disorder by administering a therapeutically effective amount of Compound 1 or a pharmaceutically acceptable salt thereof in a subject, wherein the therapeutically effective amount of Compound 1 is administered substantially contemporaneously with food.

In some embodiments, the therapeutically effective amount is in a solid dosage form. Examples of solid dosage forms include tablets, capsules, granules, powders, sachets, reconstitutable powders, dry powder inhalers and chewables.

In an aspect of the disclosure, the therapeutically effective amount of Compound 1 is administered substantially contemporaneously with food to increase the bioavailability of Compound 1, as compared to when Compound 1 is administered without food. The bioavailability of Compound 1 can be determined from plasma concentration-time curves, specifically the area under the curve (AUC). For example, bioavailability, or the increase thereof, can be determined by comparing the AUC₀₋ₜ (the AUC from the time of dosing to the last quantifiable concentration) for a formulation of Compound 1 administered with food, with the AUC₀₋ₜ for a formulation of Compound 1 without food.

In some embodiments, the therapeutically effective amount of Compound 1 (or a pharmaceutically acceptable salt thereof) is administered substantially contemporaneously with food to increase the bioavailability of Compound 1 for the treatment of a CNS related disorder, and the therapeutically effective amount of Compound 1 is in a solid dosage form such as a tablet or capsule. In some embodiments, the therapeutically effective amount of Compound 1 is administered substantially contemporaneously with food, and the bioavailability is increased by about 10% or greater; by about 20% or greater; by about 30% or greater; by about 40% or greater; by about 50% or greater; or by about 55% or greater when compared to the bioavailability when Compound 1 is administered without food.

In some embodiments, the therapeutically effective amount of Compound 1 is administered once daily substantially contemporaneously with food. In other embodiments, the therapeutically effective amount of Compound 1 is administered twice daily substantially contemporaneously with food. In some embodiments, the therapeutically effective amount of Compound 1 is administered once daily as one capsule substantially contemporaneously with food. In some embodiments, the therapeutically effective amount of Compound 1 is administered once daily as two capsules substantially contemporaneously with food.

In some embodiments, the therapeutically effective amount of Compound 1 is about 20 mg to about 60 mg and is administered substantially contemporaneously with food. In other embodiments, the therapeutically effective amount of Compound 1 is about 25 mg to about 50 mg and is administered substantially contemporaneously with food. In other embodiments, the therapeutically effective amount of Compound 1 is about 25 mg to about 35 mg and is administered substantially contemporaneously with food. In other embodiments, the therapeutically effective amount of Compound 1 is about 30 mg, e.g., 30 mg and is administered substantially contemporaneously with food.

In an aspect of the disclosure, the therapeutically effective amount of Compound 1 is administered substantially contemporaneously with food, e.g., within about 60 minutes before or after ingesting food. In some embodiments, the therapeutically effective amount of Compound 1 is administered substantially contemporaneously with food, e.g., within about 45 minutes, within about 30 minutes, within about 15 minutes, or within about 5 minutes before or after ingesting food. In some embodiments, the food is a high fat meal, as defined herein, or the food is a regular meal, as discussed herein. In other embodiments, the food is at least 50 calories, at least 100 calories, at least 200 calories, or at least 300 calories.

The disclosure features, inter alia, a method comprising administering to a subject a therapeutically effective amount of Compound 1, as described below, to treat a CNS-related disorder, e.g. a mood disorder or a movement disorder. The disclosure features methods of treating a subject having a CNS-related disorder, e.g., tremor, e.g., essential tremor; depression, e.g., postpartum depression; and anxiety disorder a composition described herein comprising Compound 1. In some embodiments, Compound 1 is administered substantially contemporeously with food or without food. In some embodiments of any of the foregoing, Compound 1 is administered substantially contemporeously with food. The disclosure also features, inter alia, such as a GABA related disease or disorder, e.g., a mood disorder, a movement disorder, postpartum depression, major depressive disorder, essential tremor, or Parkinson's disease.

In some embodiments, the CNS related disorder is a depressive disorder such as major depressive disorder. In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with Compound 1) is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with Compound 1 is greater than or equal to 23. In some embodiments, the baseline score is at least 10, 15, or 20. In some embodiments, the HAM-D total score of the subject after treatment with Compound 1 is about 0 to 10 (e.g., less than 10; 0 to 10, 0 to 6, 0 to 4, 0 to 3, 0 to 2, or 1.8). In some embodiments, the HAM-D total score after treatment with Compound 1 is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with Compound 1. In some embodiments, the decrease in the baseline HAM-D total score to HAM-D total score after treatment with Compound 1 is at least 1, 2, 3, 4, 5, 7, 10, 25, 40, 50, or 100 fold). In some embodiments, the percentage decrease in the baseline HAM-D total score to HAM-D total score after treatment with Compound 1 is at least 50% (e.g., 60%, 70%, 80%, or 90%). In some embodiments, the therapeutic effect is measured as a decrease in the HAM-D total score after treatment with Compound 1 relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with Compound 1. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with Compound 1. In some embodiments, the therapeutic effect is a decrease from baseline in HAM-D total score after treatment with Compound 1 (e.g., treatment with Compound 1 once a day for 14 days). In some embodiments, the HAM-D total score of the subject before treatment with Compound 1 is at least 24. In some embodiments, the HAM-D total score of the subject before treatment with Compound 1 is at least 18. In some embodiments, the HAM-D total score of the subject before treatment with Compound 1 is between and including 14 and 18. In some embodiments, the decrease in HAM-D total score after treating the subject with Compound 1 relative to the baseline HAM-D total score is at least 10. In some embodiments, the decrease in HAM-D total score after treating the subject with Compound 1 relative to the baseline HAM-D total score is at least 15 (e.g., at least 17). In some embodiments, the HAM-D total score associated with treating the subject with Compound 1 is no more than a number ranging from 6 to 8. In some embodiments, the HAM-D total score associated with treating the subject with Compound 1 is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Asberg Depression Rating Scale (MADRS)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

The disclosure features, inter alia, an article of manufacture comprising a therapeutically effective amount of a Compound 1, packaging material, and a label affixed to the packaging material indicating that the therapeutically effective amount of the compound should be taken with food, or a package insert contained within the packaging material indicating that the therapeutically effective amount of Compound 1 should be taken with food. In some embodiments, the therapeutically effective amount of Compound 1 is in a solid dosage form, such as a tablet or a capsule. In some embodiments, the label or package insert further indicates that the therapeutically effective amount of Compound 1 is administered once daily. In some embodiments, the label or package insert further indicates that the therapeutically effective amount of Compound 1 is about 20 mg to about 60 mg; is about 25 mg to about 50 mg; or is about 25 mg to about 35 mg; is about 30 mg, e.g., 30 mg. In some embodiments, the label or package insert further indicates that the therapeutically effective amount of Compound 1 should be taken with food that is a high fat meal or is a regular meal. In some embodiments, the label or package insert further indicates that the therapeutically effective amount of Compound 1 should be taken with food to increase the bioavailability of Compound 1. In some embodiments, the label or package insert further indicates that the therapeutically effective amount of Compound 1 is for treating CNS related disorders.

In some embodiments, the method comprises a method of treating a CNS related disorder by administering a therapeutically effective amount of Compound 1, wherein the therapeutically effective amount of Compound 1 is about 30 mg, e.g., 30 mg, and is administered substantially contemporaneously with food.

In an aspect of the disclosure, provided is, inter alia, a method for effecting positive allosteric modulation of a GABA_{A} receptor in a patient in need thereof, where the method comprises administering to the patient a therapeutically effective amount of a Compound 1 substantially contemporaneously with food. In some embodiments the therapeutically effective amount is in a solid dosage form, such as a tablet or a capsule. In some embodiments, the therapeutically effective amount of a Compound 1 is administered once daily. In some embodiments, the therapeutically effective amount of Compound 1 is about 20 mg to about 60 mg; is about 25 mg to about 50 mg; or is about 25 mg to about 35 mg; or is about 30 mg, e.g., 30 mg. In some embodiments, the therapeutically effective amount of Compound 1 is administered substantially contemporaneously with food, e.g., within about 45 minutes, within about 30 minutes, within about 15 minutes, or within about 5 minutes before or after ingesting food. In some embodiments, the food is a high fat meal, as defined herein, or the food is a regular meal, as discussed herein. In other embodiments, the food is at least 50 calories, at least 100 calories, at least 200 calories, or at least 300 calories. In some embodiments, administering the therapeutically effective amount of Compound 1 contemporaneously with food increases the bioavailability of the compound compared to administration without food. In some embodiments, bioavailability is based on a comparison of AUC values. In some embodiments, the bioavailability is increased by about 10% or greater; by about 20% or greater; by about 30% or greater; by about 40% or greater; by about 50% or greater; or by about 55% or greater. In an aspect of the disclosure, provided is, inter alia, a method of treating a CNS-related disorder, e.g., a GABA related disease or disorder, e.g., a mood disorder, a movement disorder, postpartum depression, major depressive disorder, essential tremor, or Parkinson's disease, in a subject, the method comprising administering to the subject a therapeutically effective amount of Compound 1 substantially contemporaneously with food, wherein the administration results in an increase in the bioavailability of Compound 1 compared to administration without food. In some embodiments, bioavailability is based on a comparison of AUC values. In some embodiments, bioavailability is based on the AUC₀₋ₜ value. In some embodiments, the bioavailability or AUC₀₋ₜ value for Compound 1 in a formulation is compared to the AUC₀₋ₜ value for Compound 1 in another formulation. In some embodiments, the formulation is Compound 1 in a capsule administered with food and the other formulation is Compound 1 in a capsule administered without food. In some embodiments, these AUC₀₋ₜ values are compared and the AUC₀₋ₜ value for the formulation of Compound 1 in a capsule administered with food is higher than the AUC₀₋ₜ value for the formulation of Compound 1 in a capsule administered without food. In some embodiments, the AUC₀₋ₜ value for the formulation of Compound 1 in a capsule administered with food has a percent increase of at least 10% or greater when compared to the AUC₀₋ₜ value for the formulation of Compound 1 in a capsule administered without food. In other embodiments, this percent increase is at least 20% or greater; is at least 30% or greater; is at least 40% or greater; is at least 40% or greater; or is at least 55% or greater.

In some embodiments, the CNS related disorder is a depressive disorder, e.g., major depressive disorder. In some embodiments, the method of treating the depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less.

In some embodiments, the CNS related disorder is a depressive disorder such as major depressive disorder. In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with Compound 1 is greater than or equal to 23. In some embodiments, the baseline score is at least 10, 15, or 20. In some embodiments, the HAM-D total score of the subject after treatment with Compound 1 is about 0 to 10 (e.g., less than 10; 0 to 10, 0 to 6, 0 to 4, 0 to 3, 0 to 2, or 1.8). In some embodiments, the HAM-D total score after treatment with Compound 1 is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with Compound 1. In some embodiments, the decrease in the baseline HAM-D total score to HAM-D total score after treatment with Compound 1 is at least 1, 2, 3, 4, 5, 7, 10, 25, 40, 50, or 100 fold). In some embodiments, the percentage decrease in the baseline HAM-D total score to HAM-D total score after treatment with Compound 1 is at least 50% (e.g., 60%, 70%, 80%, or 90%). In some embodiments, the therapeutic effect is measured as a decrease in the HAM-D total score after treatment with Compound 1 relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with Compound 1. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with Compound 1. In some embodiments, the therapeutic effect is a decrease from baseline in HAM-D total score after treatment with Compound 1 (e.g., treatment with Compound 1 once a day for 14 days). In some embodiments, the HAM-D total score of the subject before treatment with Compound 1 is at least 24. In some embodiments, the HAM-D total score of the subject before treatment with Compound 1 is at least 18. In some embodiments, the HAM-D total score of the subject before treatment with Compound 1 is between and including 14 and 18. In some embodiments, the decrease in HAM-D total score after treating the subject with Compound 1 relative to the baseline HAM-D total score is at least 10. In some embodiments, the decrease in HAM-D total score after treating the subject with Compound 1 relative to the baseline HAM-D total score is at least 15 (e.g., at least 17). In some embodiments, the HAM-D total score associated with treating the subject with Compound 1 is no more than a number ranging from 6 to 8. In some embodiments, the HAM-D total score associated with treating the subject with Compound 1 is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Asberg Depression Rating Scale (MADRS)) within 14, 10, 4, 3, 2, or 1 days, or 24, 20, 16, 12, 10, or 8 hours or less. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

In some embodiments of any of the foregoing, the subject is administered Compound 1, e.g., about a 30 mg dose of Compound 1, e.g., a 30 mg dose of Compound 1, once a day for less than 2 weeks. In some embodiments of any of the foregoing, the subject is administered Compound 1, e.g., about a 30 mg dose of Compound 1, e.g., a 30 mg dose of Compound 1, once a day for 1 day. In some embodiments of any of the foregoing, the subject is administered Compound 1, e.g., about a 30 mg dose of Compound 1, e.g., a 30 mg dose of Compound 1, once a day for 2 days. In some embodiments of any of the foregoing, the subject is administered Compound 1, e.g., about a 30 mg dose of Compound 1, e.g., a 30 mg dose of Compound 1, once a day for at least 14 days. In some embodiments, the subject is administered Compound 1, e.g., about a 30 mg dose, e.g., a 30 mg dose once a day for at least 28 days. In some embodiments, the subject is administered Compound 1, e.g., about a 30 mg dose, e.g., a 30 mg dose once a day for at least 6 months. In some embodiments, the subject is administered Compound 1, e.g., about a 30 mg dose, e.g., a 30 mg dose once a day for at least 1 year. In some embodiments, the subject is administered Compound 1, e.g., about a 30 mg dose, e.g., a 30 mg dose once a day for life. In some embodiments, the subject is administered Compound 1 at night. In some embodiments, the subject is administered Compound 1 no longer than 1 hour before the subject sleeps. In some embodiments, the subject is administered Compound 1 no longer than 15 minutes before the subject sleeps. In some embodiments, Compound 1 is administered chronically.

### BRIEF DESCRIPTION OF THE DRAWINGS

FIG. 1 depicts the mean (±standard deviation) Compound 1 plasma concentrations over time by dosing condition (Linear Scale).

FIG. 2 depicts the mean (±standard deviation) Compound 1 plasma concentrations over time by dosing condition (Semi-Logarithmic Scale).

FIG. 3A depicts an exemplary XRPD pattern of Form A.

FIG. 3B depicts an exemplary unit cell of Form A along the b axis.

FIG. 3C depicts exemplary TGA (upper) and DSC (lower) curves of Form A.

FIG. 3D depicts an overlay of exemplary VT-XRPD patterns of Form A at selected temperatures, along with an exemplary XRPD pattern of Form K.

FIG. 3E depicts an exemplary DVS isotherm of Form A at 25° C.

FIG. 3F depicts an exemplary XRPD patterns of Form A before and after an exemplary DVS measurement at 25° C.

FIG. 4A depicts an exemplary XRPD pattern of Form C.

FIG. 4B depicts an exemplary unit cell of Form C along the b axis.

FIG. 4C depicts exemplary TGA (upper) and DSC (lower) curves of Form C.

FIG. 4D depicts an overlay of exemplary XRPD patterns of Form C at selected temperatures as well as an exemplary XRPD pattern of Form K.

FIG. 4E depicts an overlay of exemplary XRPD patterns of Form C at selected temperatures in the presence or absence of an N2 atmosphere.

FIG. 4F depicts an exemplary DVS isotherm of Form C at 25° C.

FIG. 4G depicts an overlay of exemplary XRPD patterns of Form C before and after a DVS measurement at 25° C.

FIG. 5 depicts an exemplary XRPD pattern of Form K.

FIG. 6 depicts an overlay of exemplary XRPD patterns indicating the time-dependent conversion of Form A to Form C in ethyl acetate at an elevated temperature in the presence of seed crystals of Form C.

FIG. 7A, FIG. 7B, and FIG. 7C depict exemplary HAM-D Mean (SE) Total Score, MADRS Mean (SE) Score and HAM-A Mean (SE) Total Score for Compound 1 in a Phase 2, Open-Label Study Evaluating Compound 1 in Subjects with Major Depressive Disorder.

FIG. 8 depicts exemplary HAM-D Response and HAM-D Remission Rate for Compound 1 in a Phase 2, Open-Label Study Evaluating Compound 1 in Subjects with Major Depressive Disorder.

### DETAILED DESCRIPTION OF THE INVENTION

The disclosure is directed to Compound 1, or 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile, which has the formula

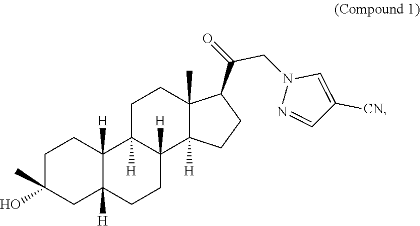

methods of using Compound 1 in the treatment of CNS related disorders, and methods for improving the effectiveness of the administration of Compound 1 for the treatment of said CNS related disorders.

In one aspect, the disclosure is directed to methods where Compound 1 can be administered to subjects in a regimen that increases the therapeutic effectiveness of Compound 1 to such subjects. Advantageously, when orally administered with food, Compound 1 exhibits increased bioavailability of Compound 1 in subjects.

It should be appreciated that in some embodiments, the described methods, e.g., methods for improving the effectiveness of the administration of Compound 1 may be directed to a pharmaceutically acceptable salt of Compound 1 for the treatment of CNS related disorders such as, but not limited to, depressive disorders, e.g., major depressive disorder.

### Definitions

The term “AUC” refers to the area under the time/plasma concentration curve after administration of the pharmaceutical composition. AUC_{0-infinity} denotes the area under the plasma concentration versus time curve from time 0 to infinity; AUC₀₋ₜ denotes the area under the plasma concentration versus time curve from time 0 to time t. As used herein, AUC₀₋ₜ is the area under the plasma concentration versus time curve from the time of dosing to the last quantifiable concentration. It should be appreciated that AUC values can be determined by known methods in the art.

As used herein, the term “bioavailability” generally means the rate and extent to which the active ingredient, or active form thereof, is absorbed from a drug product and becomes available at the site of action. See U.S. Code of Federal Regulations, Title 21, Part 320.1 (2001 ed.). For oral dosage forms, bioavailability relates to the processes by which the active ingredient is released from the oral dosage form, e.g., a tablet, converted to the active form (if the active ingredient is not already the active form), and moved to the site of action, e.g., absorbed into the systemic circulation. For example, bioavailability is based on the area under the plasma concentration-time curves (e.g., AUC₀₋ₜ). To compare the bioavailability between different formulations comprising Compound 1, the AUC₀₋ₜ values of each formulation would be compared (e.g., a comparison between a formulation of Compound 1 in a capsule administered with food and a formulation of Compound 1 in a capsule administered without food). It should be appreciated that AUC values may be compared as percent increase or percent decrease. It should further be appreciated that percent increase or percent decrease is calculated as known in the art.

The terms “without food” or “fasted” are defined to mean the condition of not having consumed food within the time period of about 2 hours prior to the administration of Compound 1 to about 2 hours after the administration of Compound 1.

As used herein, the term “unit dosage form” is defined to refer to the form in which Compound 1 is administered to the subject. Specifically, the unit dosage form can be, for example, a pill, capsule, or tablet. Preferably, the unit dosage form is a capsule. The typical amount of Compound 1 in a unit dosage form useful in the invention is about 10 mg to about 100 mg, preferably about 20 mg to about 50 mg (e.g., about 30 mg, e.g., 30 mg). In a preferred embodiment of the invention, the unit dosage form comprises about 30 mg, e.g., 30 mg, of Compound 1 and is in the form of a capsule. Preferably, capsules which comprise about 30 mg, e.g., 30 mg, of Compound 1, is administered to a subject once per day. In some embodiments, two capsules together comprise the 30 mg of Compound 1. In some embodiments, one capsule comprises the 30 mg of Compound 1.

As used herein, “substantially contemporaneously with food” or “substantially contemporaneous” means ingesting (or introducing) a substance containing food (e.g., high fat meal, a standard meal or a regular meal, food comprising at least 50 calories, food comprising at least 100 calories, food comprising at least 200 calories, or food comprising at least 300 calories) within 5, 10, 15, 30, 45, 60, 75, or 90 minutes before or after ingesting (or introducing) a composition of the invention, e.g., a therapeutically effective amount of Compound 1.

The term “Cₘₐₓ” refers to the maximum concentration of a therapeutic agent (e.g. Compound 1) in the blood (e.g. plasma) following administration of the pharmaceutical composition.

The term “tₘₐₓ” refers to the time in hours when Cₘₐₓ is achieved following administration of the pharmaceutical composition comprising the therapeutic agent (e.g. Compound 1).

As used herein, “solid dosage form” means a pharmaceutical dose(s) in solid form, e.g. tablets, capsules, granules, powders, sachets, reconstitutable powders, dry powder inhalers and chewables.

As used herein, “high fat meal” means a high fat and high calorie meal with approximately 50 percent of total caloric content of the meal from fat and the meal being approximately 800 to 1000 calories. The meal may also be approximately 150, 250, and 500-600 calories from protein, carbohydrate, and fat, respectively. An exemplary high fat meal includes the test meal disclosed in the document Guidance for Industry, Food-Effect Bioavailability and Fed Bioequivalence Studies, U.S. Department of Health and Human Services Food and Drug Administration, Center for Drug Evaluation and Research (CDER), Center for Biologics Evaluation and Research (CBER) issued December 2002. The exemplary high-fat meal contains approximately 50 percent of the total caloric content of the meal as fat and contains approximately 800 to 1000 calories; 500-600 calories from fat. As used herein, the term “fat” is used in its conventional, art-recognized meaning.

As used herein, “regular meal” or “standard meal” means a meal being approximately 300 to 800 calories.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

As used herein, the “baseline” total score of a scale described herein, e.g., the Hamilton Depression scale, Montgomery-Asberg Depression Rating Scale, or Clinical Global Impression-Improvement Scale, used to evaluate a subject for a disorder, e.g., a depressive disorder, e.g., major depressive disorder, is the determined total score of the subject prior to treatment with a therapeutic, e.g., Compound 1, to treat said disorder.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

The articles “a” and “an” may be used herein to refer to one or to more than one (i.e. at least one) of the grammatical objects of the article. By way of example “an analogue” means one analogue or more than one analogue.

As used herein, the term “modulation” refers to the inhibition or potentiation of GABA receptor function. A “modulator” (e.g., a modulator compound) may be, for example, an agonist, partial agonist, antagonist, or partial antagonist of the GABA receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, and unless otherwise specified, a “cycle of treatment” comprises administering a first dose of a neuroactive steroid, administering a second dose of the neuroactive steroid, and administering a third dose of the neuroactive steroid, said neuroactive steroid doses being sufficient to treat said subject.

In general, the “effective amount” of a compound refers to an amount sufficient to elicit the desired biological response, e.g., to treat a CNS-related disorder, e.g., a disorder as described herein (e.g., tremor (e.g., essential tremor); depression (e.g., postpartum depression); or an anxiety disorder). As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, “crystalline” refers to a solid having a highly regular chemical structure, i.e., having long range structural order in the crystal lattice. The molecules are arranged in a regular, periodic manner in the 3-dimensional space of the lattice. In particular, a crystalline form may be produced as one or more single crystalline forms. For the purposes of this application, the terms “crystalline form”, “single crystalline form,” “crystalline solid form,” “solid form,” and “polymorph” are synonymous and used interchangably; the terms distinguish between crystals that have different properties (e.g., different XRPD patterns and/or different DSC scan results).

The term “substantially crystalline” refers to forms that may be at least a particular weight percent crystalline. Particular weight percentages are 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, Compound 1 can be a substantially crystalline sample of any of the crystalline solid forms described herein (e.g., Forms, A, C, or K).

The term “substantially pure” relates to the composition of a specific crystalline solid form of Compound 1 that may be at least a particular weight percent free of impurities and/or other solid forms of Compound 1. Particular weight percentages are 70%, 75%, 80%, 85%, 90%, 95%, 99%, or any percentage between 70% and 100%. In some embodiments, Compound 1 can be a substantially pure sample of any of the crystalline solid forms described herein. (e.g., Forms, A, C, or K,). In some embodiments, Compound 1 can be substantially pure Form A. In some embodiments, Compound 1 can be substantially pure Form C.

As used herein, the term “anhydrous” or “anhydrate” when referring to a crystalline form of Compound 1 means that no solvent molecules, including those of water, form a portion of the unit cell of the crystalline form. A sample of an anhydrous crystalline form may nonetheless contain solvent molecules that do not form part of the unit cell of the anhydrous crystalline form, e.g., as residual solvent molecule left behind from the production of the crystalline form. In a preferred embodiment, a solvent can make up 0.5% by weight of the total composition of a sample of an anhydrous form. In a more preferred embodiment, a solvent can make up 0.2% by weight of the total composition of a sample of an anhydrous form. In some embodiments, a sample of an anhydrous crystalline form of Compound 1 contains no solvent molecules, e.g., no detectable amount of solvent. The term “solvate” when referring to a crystalline form of Compound 1 means that solvent molecules, e.g., organic solvents and water, form a portion of the unit cell of the crystalline form. Solvates that contain water as the solvent are also referred to herein as “hydrates.” The term “isomorphic” when referring to a crystalline form of Compound 1 means that the form can comprise different chemical constituents, e.g., contain different solvent molecules in the unit cell, but have identical XRPD patterns. Isomorphic crystalline forms are sometimes referred to herein as “isomorphs.”

The term “characteristic peaks” when referring to the peaks in an XRPD pattern of a crystalline form of Compound 1 refers to a collection of certain peaks whose values of 20 across a range of 0°-40° are, as a whole, uniquely assigned to one of the crystalline forms of Compound 1.

### Bioavailability and Food

Food can change the bioavailability of a drug or compound and can have clinically significant consequences. Food can alter bioavailability in an unpredictable manner by various means, including delay gastric emptying, stimulate bile flow, change gastrointestinal (GI) pH, increase splanchnic blood flow, change luminal metabolism of a drug substance, and/or physically and chemically interact with a dosage form or a drug substance. The nutrient and caloric contents of the meal, the meal volume, and the meal temperature can cause physiological changes in the GI tract in a way that affects drug product transit time, luminal dissolution, drug permeability, and systemic availability. Administration of a drug or compound with food may change the bioavailability by affecting either the drug substance or the drug product. It is difficult to determine the mechanism by which food changes the bioavailability of a drug or compound.

The disclosure provides a method of increasing the extent of absorption of Compound 1 as measured by the concentration attained in the blood stream over time in a subject in need of a therapeutic effect thereof. This method comprises orally administering to a subject a therapeutically effective amount of Compound 1 with food. The concentration in the blood stream is measured as the plasma concentration (e.g., ng/mL) of Compound 1. Pharmacokinetic parameters involved in determining the plasma concentration include the maximum observed plasma concentration (Cₘₐₓ), area under the plasma concentration time curve (AUC) from time zero up to the last quantifiable concentration (AUC₀₋ₜ), and AUC from time zero to infinity (AUC_{0-∞}). Administering Compound 1 to a subject with food increases the bioavailability as measured by increased values of one or more of the aforesaid pharmacokinetic parameters, when compared to administration of the drug under fasted (or without food) conditions. In some embodiments, the AUC₀₋ₜ value for the formulation of Compound 1 in a capsule administered with food has a percent increase of at least 10% or greater when compared to the AUC₀₋ₜ value for the formulation of Compound 1 in a capsule administered without food. In other embodiments, this percent increase is at least 20% or greater; is at least 30% or greater; is at least 40% or greater; is at least 40% or greater; or is at least 55% or greater.

A subject may take a compound or drug in a fasted state or a fed state, or with food or without food. In a fasted state, the subject may fast, for example overnight, for at least ten (10) hours. The subject may then take the drug or compound, with the subject taking no food for at least four hours post-dose. Additionally, the subject may fast, for example, for two hours and then take the drug or compound, with the subject taking no food for at least two hours post-dose. In a fed state, or taking the drug with food, the subject may start the meal (high fat or regular) thirty (30) minutes prior to the administration of the drug or compound. The subject may then eat this meal in 30 minutes or less; however, the drug or compound may be administered thirty (30) minutes after start of the meal. Thus, it is desired to administer a compound, such as Compound 1 in such a manner that bioavailability would be maximized.

One aspect of the disclosure is a method of increasing the bioavailability of Compound 1 in a subject by administering Compound 1 in a therapeutically effective amount with food. In one aspect of the disclosure, the subject is administered a therapeutically effective amount of Compound 1 substantially contemporaneously with food. The food may be a high fat meal or a regular meal. A high fat meal may comprise about 50 percent of the total caloric content of the meal as fat and about 800 to 1000 calories. An exemplary high fat meal includes the test meal disclosed in the document Guidance for Industry, Food-Effect Bioavailability and Fed Bioequivalence Studies, U.S. Department of Health and Human Services Food and Drug Administration, Center for Drug Evaluation and Research (CDER), Center for Biologics Evaluation and Research (CBER) issued December 2002. The exemplary high-fat meal contains approximately 50 percent of the total caloric content of the meal as fat and contains approximately 800 to 1000 calories; 500-600 calories from fat. As used herein, the term “fat” is used in its conventional, art-recognized meaning. For example, a high fat meal may be two eggs fried in butter, two strips of bacon, two slices of toast with butter, 4 oz. of hash brown potatoes and 8 oz. of whole milk. A regular meal or a standard meal may be a meal such as breakfast, lunch or dinner having calories of 300 to 800 calories. For example, a subject could eat dinner, finish the dinner within 30 minutes, and then take (ingest) the therapeutically effective amount of Compound 1 promptly after finishing the dinner.

Methods of the disclosure include administering a therapeutically effective amount of Compound 1 substantially contemporaneously with food, wherein the food may be a snack, or less than a meal. For example, Compound 1 may be administered substantially contemporaneously with food, where the food contains about 50 calories. Additionally, Compound 1 may be administered substantially contemporaneously with food, where the food contains about 100 calories. Compound 1 may be administered substantially contemporaneously with food, where the food contains about 200 or about 300 calories. For example, a subject could ingest a food (e.g. snack) such as fruit, granola, crackers, cheese, etc., and then the subject would take (ingest) a therapeutically effective amount of Compound 1.

In another aspect of the disclosure, a subject is administered a therapeutically effective amount of Compound 1 substantially contemporaneously with food (e.g., a high fat, regular meal, or food containing about 50 to about 300 calories, as discussed herein), where substantially contemporaneously with food means administering the therapeutically effective amount of Compound 1 within 5, 10, 15, 30, 45, 60, 75, or 90 minutes before or after ingesting or eating the food. For example, administering a therapeutically effective amount of Compound 1 within approximately 90 minutes before or after ingesting or eating the food (e.g., a high fat, regular meal, or food containing about 50 to about 300 calories, as discussed herein). For example, administering a therapeutically effective amount of Compound 1 within approximately 75 minutes before or after ingesting or eating the food (e.g., a high fat, regular meal, or food containing about 50 to about 300 calories, as discussed herein). For example, administering a therapeutically effective amount of Compound 1 within approximately 60 minutes before or after ingesting or eating the food (e.g., a high fat, regular meal, or food containing about 50 to about 300 calories, as discussed herein). For example, administering a therapeutically effective amount of Compound 1 within approximately 45 minutes before or after ingesting or eating the food (e.g., a high fat, regular meal, or food containing about 50 to about 300 calories, as discussed herein). For example, administering a therapeutically effective amount of Compound 1 within approximately 30 minutes before or after ingesting or eating the food (e.g., a high fat, regular meal, or food containing about 50 to about 300 calories, as discussed herein). For example, administering a therapeutically effective amount of Compound 1 within approximately 15 minutes before or after ingesting or eating the food (e.g., a high fat, regular meal, or food containing about 50 to about 300 calories, as discussed herein). For example, administering a therapeutically effective amount of Compound 1 within approximately 10 minutes before or after ingesting or eating the food (e.g., a high fat, regular meal, or food containing about 50 to about 300 calories, as discussed herein). For example, administering a therapeutically effective amount of Compound 1 within approximately 5 minutes before or after ingesting or eating the food (e.g., a high fat, regular meal, or food containing about 50 to about 300 calories, as discussed herein).

Compound 1 may be administered any time of day with food, for example, any time of day substantially contemporaneously with food (e.g., a high fat, regular meal, or food containing about 50 to about 300 calories, as discussed herein). For example, a subject may be administered (or self-administer) a therapeutically effective amount of Compound 1 substantially contemporaneously with breakfast. For example, a subject may be administered (or self-administer) a therapeutically effective amount of Compound 1 substantially contemporaneously with lunch. For example, a subject may be administered (or self-administer) a therapeutically effective amount of Compound 1 substantially contemporaneously with dinner.

The increased bioavailability of Compound 1 to a subject receiving Compound 1 can be evidenced in any suitable manner. Desirably, the oral administration of Compound 1 with food results in an increased bioavailability of Compound 1 as evidenced by an increase in the AUC₀₋ₜ value of Compound 1 as compared to the AUC₀₋ₜ value of Compound 1 without food.

### Articles of Manufacture

Compositions of the disclosure may also be packaged as articles of manufacture comprising a therapeutically effective amount of Compound 1. Any of the various methods known by persons skilled in the art for packaging tablets, caplets, or other solid dosage forms suitable for oral administration, that will not degrade the components of the present disclosure, are suitable for use in packaging.

In some aspects, an article of manufacture comprises a therapeutically effective amount of Compound 1, packaging material, and a label affixed to the packaging material or a package insert contained within the packaging material.

In some embodiments, the packaging material comprises at least one container. In some embodiments, the packaging material comprises multiple containers. As used herein, a container is an object that holds the therapeutically effective amount of Compound 1. For example, the container may be a bottle, a blister pack, a box, a carton, a strip package, a cartridge, or a single-dose container. In some embodiments, the container is a bottle that holds a therapeutically effective amount of Compound 1. In some embodiments, a box contains the bottle that holds the therapeutically effective amount of Compound 1. In some embodiments, the container is a blister pack that holds the therapeutically effective amount of Compound 1.

It should be appreciated that the packaging material may comprise a single material or various materials. For example, the packaging material may be comprised of glass, paper, plastic or metal materials. In some embodiments, the packaging material is composed of glass, plastic and metal materials. In some embodiments, the packaging material is composed of glass and plastic. In some embodiments, the packaging material is composed of glass and metal materials. In some embodiments, the packaging material is composed of plastic and metal materials. In some embodiments, the packaging material is composed of glass materials. For example, the packaging material is a glass bottle. In some embodiments, the packaging material is composed of plastic materials. For example, the packaging material is a plastic bottle or a plastic blister pack. In some embodiments, the packaging material is composed of metal materials. For example, the packaging material is a metal (e.g., aluminum) blister pack.

In some embodiments, the container has a closure. Closures are used for the purpose of covering drug containers after filling the container with solid dosage forms comprising Compound 1. Depending on the type of container, closures may have different shapes and sizes. A closure may be rubber, may be a cap or overseal, may be a tamper-evident closure, may be a child-resistant closure, etc. A packaging material of the disclosure may have one, two, three, four or five types of closure. For example, if the container is a glass bottle, the glass bottle may have a rubber seal and a plastic cap.

The packaging material may also have labelling and information related to the pharmaceutical composition printed thereon. Additionally, an article of manufacture may contain a brochure, report, notice, pamphlet, or leaflet containing product information. This form of pharmaceutical information is referred to in the pharmaceutical industry as a “package insert.” A package insert may be attached to or included with an article of manufacture. The package insert and any article of manufacture labelling provides information relating to the therapeutically effective amount of Compound 1. The information and labelling provides various forms of information utilised by health-care professionals and patients, describing the therapeutically effective amount of Compound 1, its dosage and various other parameters required by regulatory agencies such as the United States Food and Drug Agencies.

Compound 1 desirably is provided to a subject in an article of manufacture, associated with prescribing information that advises the subject to orally administer Compound 1 with food. The article of manufacture may also explain that doing so will increase the bioavailability of Compound 1. Compound 1 preferably is provided to a subject in an article of manufacture, associated with prescribing information that advises the subject that the administration of the dose of Compound 1 with food results in an increase in the extent of absorption of Compound 1 as reflected by an increase in the AUC₀₋ₜ value of Compound 1 as compared to the administration of the drug under fasted conditions. In some embodiments, Compound 1 is in packaging material with a label affixed to the packaging material indicating that the therapeutically effective amount of Compound 1 should be taken with food or a package insert contained within the packaging material indicating that the therapeutically effective amount of Compound 1 should be taken with food. The labeling instructions will be consistent with the methods of treatment as described herein. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

### Pharmaceutical Compositions

In one aspect, the disclosure provides a pharmaceutical composition comprising a compound of the present invention (also referred to as the “active ingredient”), for example Compound 1, and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In preferred embodiments, Compound 1 is administering to a subject orally.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In some embodiments, Compound 1 is in a solid form or a crystalline form. For example, Compound 1 may be in Form A (as discussed herein), Form C (as discussed herein), or Form K (as discussed herein).

Form A can be prepared by stirring crude Compound 1 as a slurry in ethyl acetate below 10° C. and then filtering and drying under vacuum or by dissolving crude Compound 1 in dichloromethane and then re-concentrating the solution twice with ethyl acetate under vacuum to dryness. Form A can be determined to be a crystalline form of Compound 1 by XRPD. TGA, together with single-crystal structure of Form A, can be used to conclude that Form A is anhydrous. DSC can be used to indicate the presence of two endotherms occurring at temperatures below 300° C.: one endotherm with a Tonset of 157.2° C. that represents the transformation of Form A into Form K, and another with a Tonset of 203.8° C. that represents the melting point of Form K. DVS can be used to demonstrate that Form A exhibits less than 0.30 weight percent water uptake at a relative humidity (RH) less than or equal to 95%.

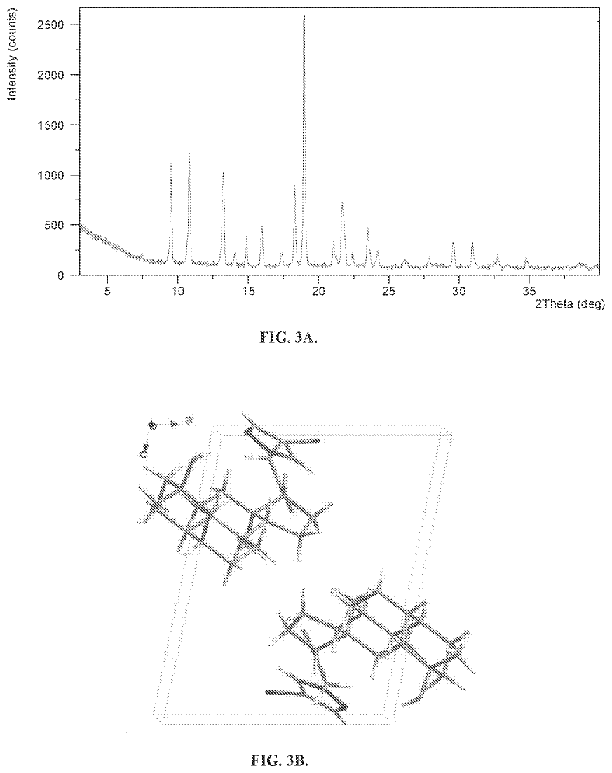

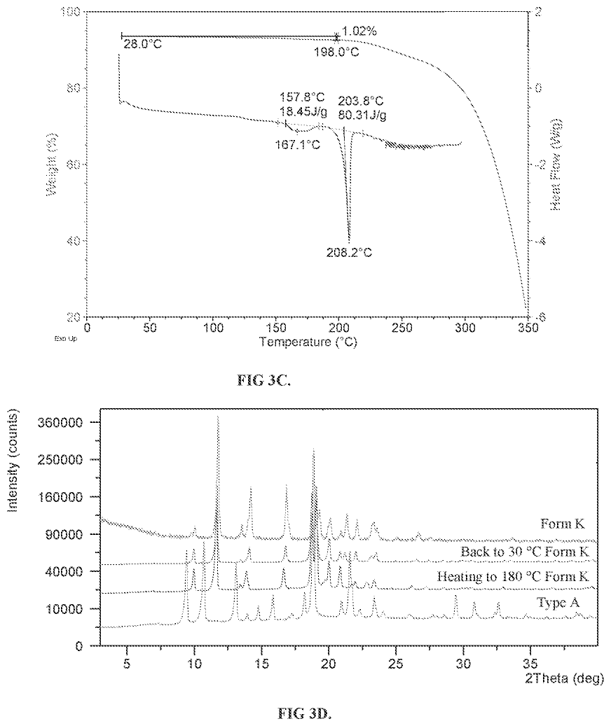

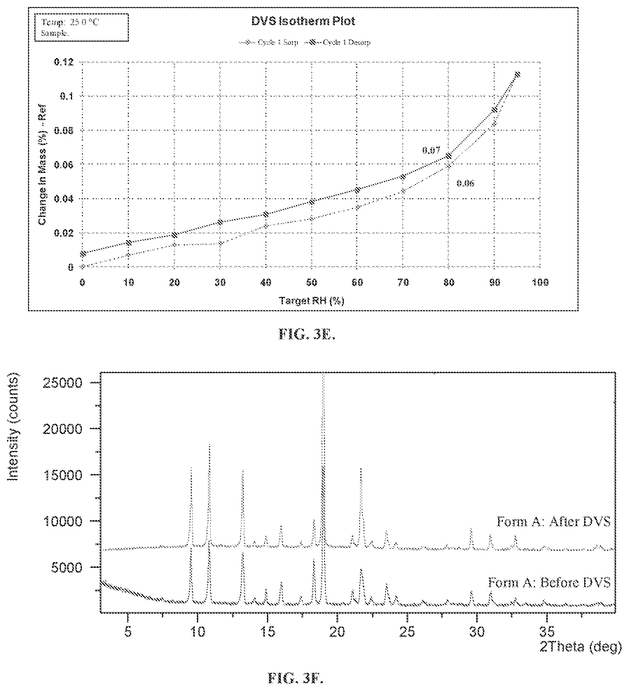

In some embodiments, Form A can have an XRPD pattern substantially as depicted in FIG. 3A. Additionally, representative peaks from the XRPD pattern of Form A can be indicated by their values of 20, d-spacing, and relative intensities as, for example, in Table 1 below:

TABLE 1
Selected experimental XRPD pattern data for Form A.

| 2θ (degrees) | d-spacing (Å) | Relative Intensity (%) |
|---|---|---|
| 9.494611 | 9.31518 | 40.49 |
| 10.78823 | 8.20093 | 46.5 |
| 13.22776 | 6.69345 | 37.69 |
| 14.89123 | 5.94927 | 10.18 |
| 15.99324 | 5.54174 | 15.09 |
| 18.28113 | 4.85302 | 31.96 |
| 18.93233 | 4.68754 | 100 |
| 21.05207 | 4.2201 | 10.38 |
| 21.64548 | 4.10573 | 24.16 |
| 23.50505 | 3.78495 | 15.37 |

In some embodiments, Form A has an XRPD pattern with characteristic peaks between and including the following values of 20 in degrees: 9.3 to 9.7 (e.g., 9.5), 10.6 to 11.0 (e.g., 10.8), 13.0 to 13.4 (e.g., 13.2), 14.7 to 15.1 (e.g., 14.9), 15.8 to 16.2 (e.g., 16.0), 18.1 to 18.5 (e.g., 18.3), 18.7 to 19.1 (e.g., 18.9), 20.9 to 21.3 (e.g., 21.1), 21.4 to 21.8 (e.g., 21.6), and 23.3 to 23.7 (e.g., 23.5). In some embodiments, Form A has an XRPD pattern with characteristic peaks between and including the following values of 20 in degrees: 9.3 to 9.7 (e.g., 9.5), 10.6 to 11.0 (e.g., 10.8), 13.0 to 13.4 (e.g., 13.2), 18.7 to 19.1 (e.g., 18.9), and 21.4 to 21.8 (e.g., 21.6). In some embodiments, Form A has an XRPD pattern with characteristic peaks at the following values of 20 in degrees: 9.5, 10.8, 13.2, 14.9, 16.0, 18.3, 18.9, 21.1, 21.6, and 23.5. In some embodiments, Form A has an XRPD pattern with characteristic peaks at the following values of 20 in degrees: 9.5, 10.8, 13.2, 18.9, and 21.6.

Calculated XRPD data for selected peaks can be obtained from X-ray diffraction data from a single crystal of Form A as provided in Table 2 below, which complement the experimental data in Table 1.

TABLE 2
Selected calculated XRPD pattern data for Form A.

| 2θ (degrees) | d-spacing (Å) | Relative Intensity (%) |
|---|---|---|
| 10.9265 | 8.09076 | 21.14 |
| 13.32097 | 6.64132 | 48.56 |
| 14.11329 | 6.27021 | 20.36 |
| 14.94619 | 5.92262 | 24.95 |
| 16.05232 | 5.5169 | 49.72 |
| 17.42404 | 5.08555 | 48.28 |
| 18.40825 | 4.81581 | 100 |
| 19.2493 | 4.60725 | 18.47 |
| 24.23572 | 3.66943 | 19.02 |
| 24.3725 | 3.64915 | 19.56 |

Form C is a crystalline anhydrate of Compound 1 as determined by XRPD and can be prepared from Form A using a slurry conversion crystallization technique in isopropyl alcohol and isopropyl acetate at 50° C. TGA and single-crystal X-ray crystallography can be used to confirm the absence of solvent in Form C. DSC can be used to indicate two endotherms below 300° C.: a broad peak with a Tonset of 183.8° C. corresponding to the transformation of Form C into Form K and a sharp peak with a Tonset of 211.0° C. corresponding to the melting of Form K. DVS can be used to demonstrate that Form C exhibits less than 0.32 weight percent water uptake at RH less than or equal to 95%.

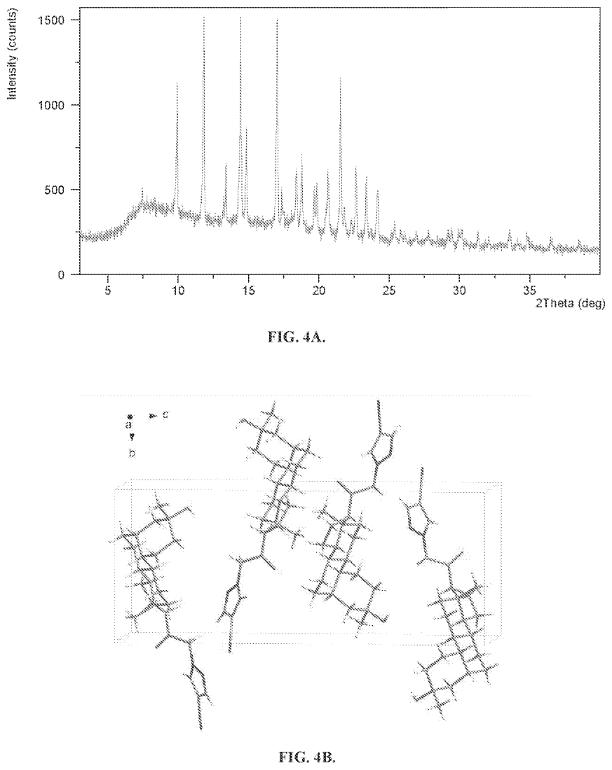

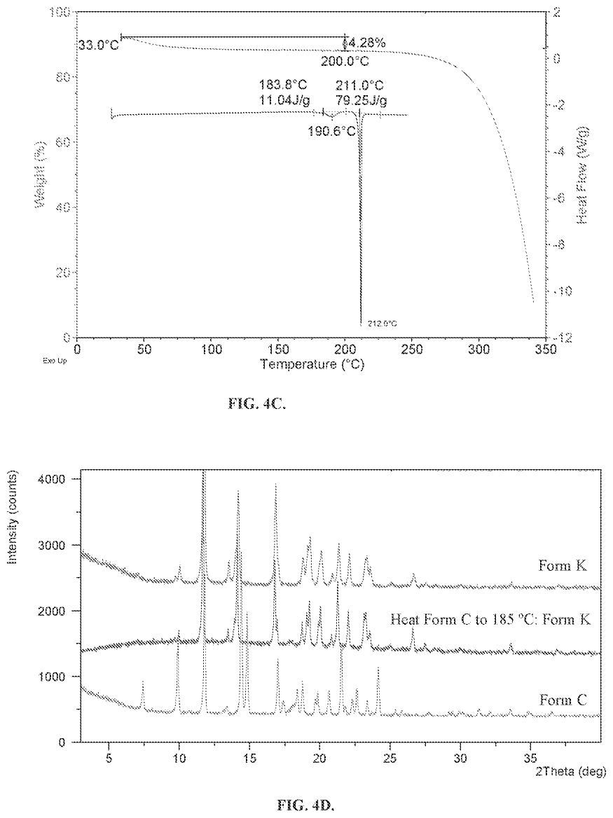

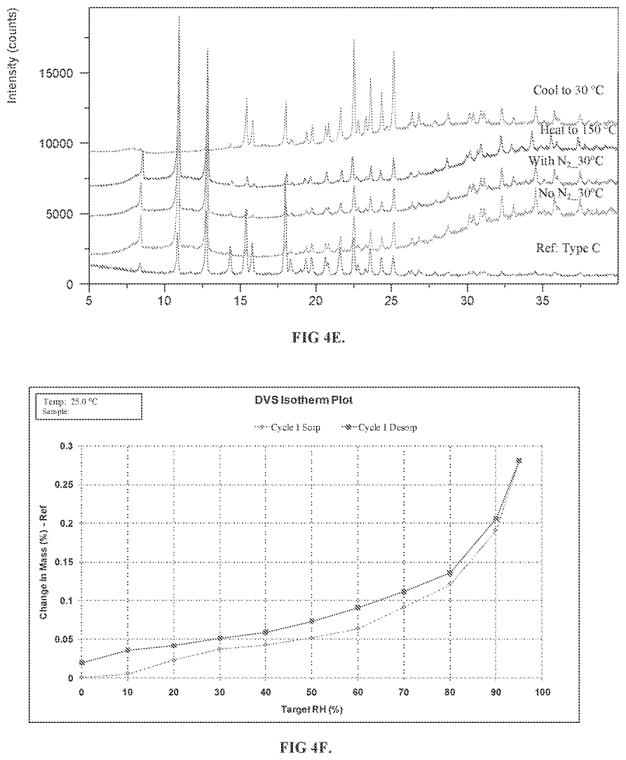

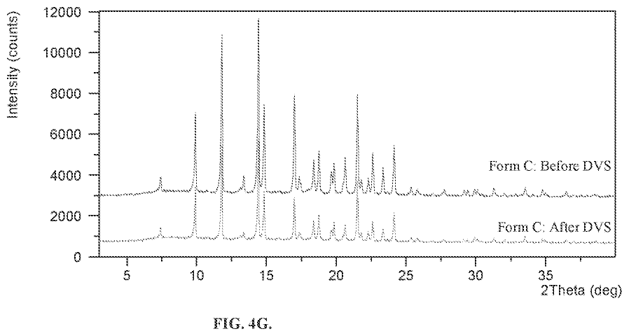

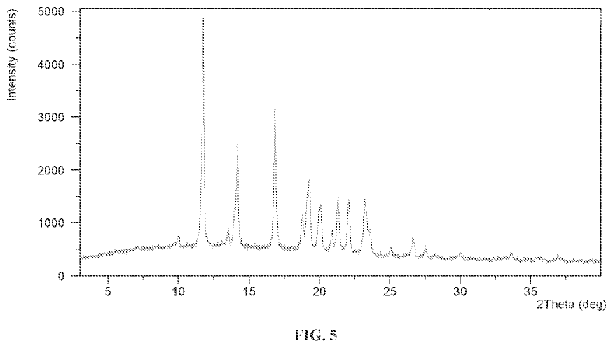

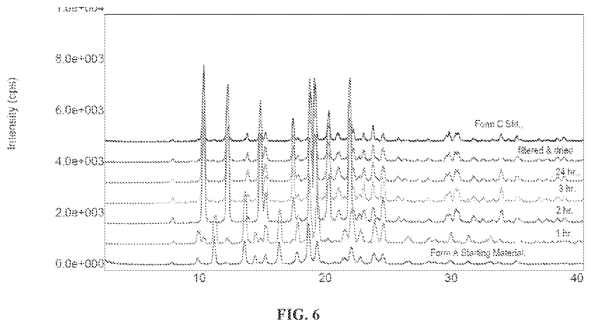

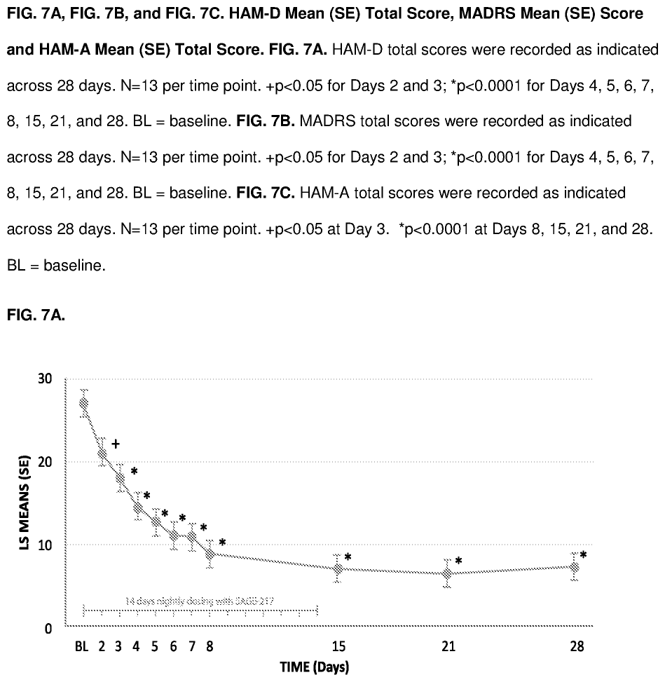

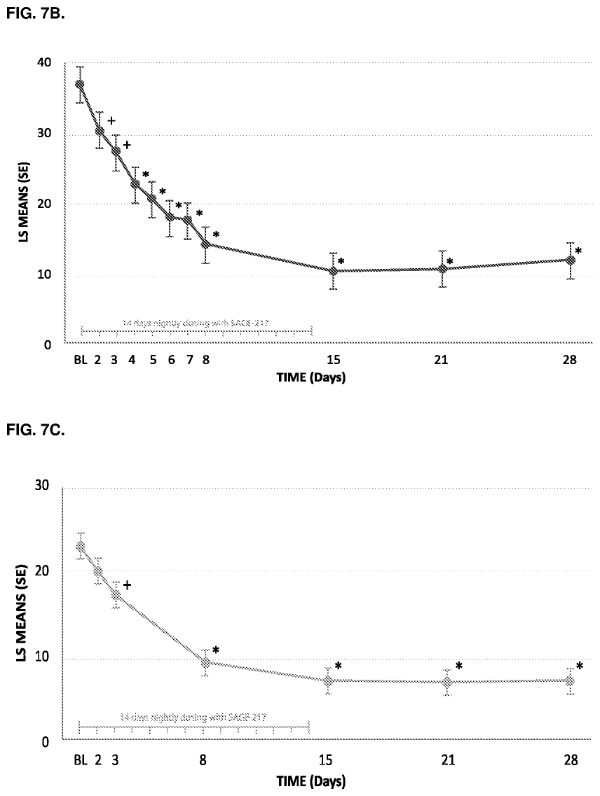

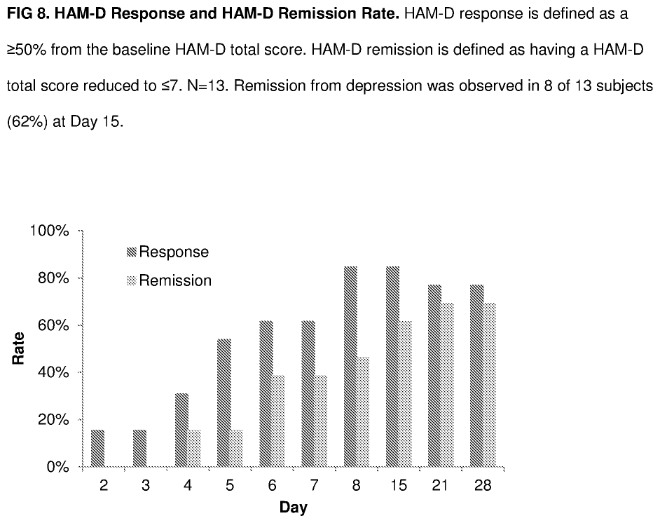

In some embodiments, Form C can have an XRPD pattern substantially as depicted in FIG. 4A. Additionally, representative peaks from the XRPD pattern of Form C can be indicated by their values of 2θ, d-spacing, and relative intensities as, for example, in Table 3 below:

TABLE 3
Selected experimental XRPD pattern data for Form C.

| 2θ (degrees) | d-spacing (Å) | Relative Intensity (%) |
|---|---|---|
| 22.60955 | 3.93279 | 26.76 |
| 20.65623 | 4.30006 | 27.84 |
| 13.36358 | 6.62573 | 28.42 |
| 14.81188 | 5.98097 | 33.78 |
| 21.50066 | 4.12963 | 36.7 |
| 21.54634 | 4.12439 | 36.94 |
| 9.889125 | 8.94443 | 41.85 |
| 11.79075 | 7.50579 | 65.73 |
| 14.41313 | 6.14552 | 65.89 |
| 16.99542 | 5.21715 | 100 |

In some embodiments, Form C can have an XRPD pattern with characteristic peaks between and including the following values of 20 in degrees: 9.7 to 10.1 (e.g., 9.9), 11.6 to 12.0 (e.g., 11.8), 13.2 to 13.6 (e.g., 13.4), 14.2 to 14.6 (e.g., 14.4), 14.6 to 15.0 (e.g., 14.8), 16.8 to 17.2 (e.g., 17.0), 20.5 to 20.9 (e.g., 20.7), 21.3 to 21.7 (e.g., 21.5), 21.4 to 21.8 (e.g., 21.6), and 22.4 to 22.8 (e.g., 22.6). In some embodiments, Form C can have an XRPD pattern with characteristic peaks between and including the following values of 2θ in degrees: 9.7 to 10.1 (e.g., 9.9), 14.6 to 15.0 (e.g., 14.8), 16.8 to 17.2 (e.g., 17.0), 20.5 to 20.9 (e.g., 20.7), and 21.3 to 21.7 (e.g., 21.5). In some embodiments, Form C can have an XRPD pattern with characteristic peaks at the following values of 2θ in degrees: 9.9, 11.8, 13.4, 14.4, 14.8, 17.0, 20.7, 21.5, 21.6, and 22.6. In some embodiments, Form C can have an XRPD pattern with characteristic peaks at the following values of 2θ in degrees: 9.9, 14.8, 17.0, 20.7, and 21.5.

Calculated XRPD data for selected peaks can be obtained using X-ray diffraction data from a single crystal of Form C, as provided in Table 4 below. These simulated peaks can complement the experimental data in Table 3.

TABLE 4
Selected calculated XRPD pattern data for Form C.

| 2θ (degrees) | d-spacing (Å) | Relative Intensity (%) |
|---|---|---|
| 9.861923 | 8.96162 | 19.41 |
| 11.75959 | 7.51938 | 37.75 |
| 13.33554 | 6.6341 | 31.9 |
| 14.38478 | 6.15248 | 43.36 |
| 14.79021 | 5.98473 | 26.68 |
| 16.96659 | 5.22162 | 100 |
| 19.61234 | 4.52277 | 17.69 |
| 20.60123 | 4.30785 | 30.39 |
| 21.48653 | 4.13232 | 25.6 |
| 22.57956 | 3.93469 | 27.32 |

Form K can be prepared by heating various forms of Compound 1, e.g., Form A or Form C to elevated temperatures. The analyzed sample of this form can be determined to be crystalline by XRPD analysis. TGA can be used to indicate no weight loss prior to the decomposition temperature and demonstrates that Form K is anhydrous. DSC can be used to demonstrate that Form K can exhibit a single endotherm with a Tonset of 211.6° C. that corresponds to the melting point of the analyzed sample. DVS measurements were performed to demonstrate that Form K demonstrates less than 0.35 weight percent water uptake at RH less than or equal to 95%.

In some embodiments, Form K can have an XRPD pattern substantially as depicted in FIG. 5. Additionally, representative peaks from the XRPD pattern of Form K can be indicated by their values of 20 and relative intensities as, for example, in Table 5 below:

Table 5. Selected experimental XRPD pattern data for Form K.

2θ (degrees) d-spacing (Å) Relative Intensity (%)

13.9471 6.3498 19.12

20.09767 4.41829 20.68

23.20826 3.83268 23.69

22.05504 4.0304 24.27

19.10905 4.64459 24.93

21.32362 4.16697 26.68

19.33614 4.59055 28.07

14.16125 6.25426 47

16.84678 5.26284 61.56

11.75077 7.53124 100

In some embodiments, Form K can have an XRPD pattern with characteristic peaks between and including the following values of 2θ in degrees: 11.6 to 12.0 (e.g., 11.8), 13.7 to 14.1 (e.g., 13.9), 14.0 to 14.4 (e.g., 14.2), 16.6 to 17.0 (e.g., 16.8), 18.9 to 19.3 (e.g., 19.1), 19.1 to 19.5 (e.g., 19.3), 19.9 to 20.3 (e.g., 20.1), 21.1 to 21.5 (e.g., 21.3), 21.9 to 22.3 (e.g., 22.1), and 23.0 to 23.4 (e.g., 23.2). In some embodiments, Form K can have an XRPD pattern with characteristic peaks between and including the following values of 2θ in degrees: 11.6 to 12.0 (e.g., 11.8), 16.6 to 17.0 (e.g., 16.8), 18.9 to 19.3 (e.g., 19.1), 19.9 to 20.3 (e.g., 20.1), and 23.0 to 23.4 (e.g., 23.2). In some embodiments, Form K can have an XRPD pattern with characteristic peaks at the following values of 2θ in degrees: 11.8, 13.9, 14.2, 16.8, 19.1, 19.3, 20.1, 21.3, 22.1, and 23.2. In some embodiments, Form K can have an XRPD pattern with characteristic peaks at the following values of 2θ in degrees: 11.8, 16.8, 19.1, 20.1, and 23.2.

Solid compositions, e.g., solid dosage forms, may include, for example, any of the following ingredients, or a solid form of Compound 1 of a similar nature: binders, surfactants, diluents or fillers, buffering agents, antiadherents, glidants, hydrophilic or hydrophobic polymers, retardants, stabilizing agents or stabilizers, disintegrants or superdisintegrants, dispersants, antioxidants, antifoaming agents, fillers, flavors, colorants, lubricants, sorbents, preservatives, plasticizers, coatings, or sweeteners, or mixtures thereof, For example, the exipeint or excipients could be a binder such as microcrystalline cellulose, polyvinyl pyrrolidone, hydroxylpropyl cellulose, low viscosity hydroxypropylmethylcellulose, gum tragacanth or gelatin; a diluent such as mannitol, microcrystalline cellulose, maltodextrin, starch or lactose, a disintegrating agent such as alginic acid, Primogel, sodium starch glycolate, sodium croscarmellose, crospovidone, or corn starch; a lubricant such as magnesium stearate, sodium stearyl fumarate or glyceryl behenate; a glidant such as colloidal silicon dioxide; a preservative such as potassium sorbate or methyl paraben, asurfactant, such as sodium lauryl sulfate, sodium docusate, poysorbate 20, polysorbate 80, cetyl triethyl ammonium bromide, polyethyelene oxide-polypropylene oxide copolymers, or Cremophor EL. an antioxidant such as butylhydroxy toluene, butyl hydroxyanisole, propyl gallate, ascorbic acid, tocopherol or tocopherol acetate, sodium sulphite, or sodium metabisulfite, a coating comprising one or more of hydroxypropykmethylcellulose, polyvinyl alcohol, methacrylate copolymers, cellulose acetate, hydroxypropylmethylcellulose acetate succinate, shellac and others, a sweetening agent such as sucrose, sucralose, acesulfame K, sodium aspartame or saccharin; or a flavoring agent such as peppermint, methyl salicylate, or orange flavoring. Any of the well-known pharmaceutical excipients may be incorporated in the dosage form and may be found in the FDA's Inactive Ingredients Guide, Remington: The Science and Practice of Pharmacy, Twenty-first Ed., (Pharmaceutical Press, 2005); Handbook of Pharmaceutical Excipients, Sixth Ed. (Pharmaceutical Press, 2009) all of which are incorporated by reference.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration and stability of the active ingredients or Formulation. All such known transdermal formulations and ingredients are included within the scope provided herein. Topical delivery compositions of interest include liquid formulations, such as lotions (liquids containing insoluble material in the form of a suspension or emulsion, intended for external application, including spray lotions) and aqueous solutions, semi-solid formulations, such as gels (colloids in which the disperse phase has combined with the dispersion medium to produce a semisolid material, such as a jelly), creams (soft solids or thick liquids) and ointments (soft, unctuous preparations), and solid formulations, such as topical patches. As such, delivery vehicle components of interest include, but are not limited to: emulsions of the oil-in-water (O/W) and the water in-oil (W/O) type, milk preparations, lotions, creams, ointments, gels, serum, powders, masks, packs, sprays, aerosols, sticks, and patches.

Compound 1 provided herein can also be administered by a transdermal device. Accordingly, transdermal administration can be accomplished using a patch either of the reservoir or membrane type, or of an adhesive matrix or other matrix variety. Delivery compositions of interest include liquid formulations, such as lotions (liquids containing insoluble material in the form of a suspension or emulsion, intended for external application, including spray lotions) and aqueous solutions, semi-solid formulations, such as gels (colloids in which the disperse phase has combined with the dispersion medium to produce a semisolid material, such as a jelly), creams (soft solids or thick liquids) and ointments (soft, unctuous preparations), and solid formulations, such as topical patches. As such, delivery vehicle components of interest include, but are not limited to: emulsions of the oil-in-water (O/W) and the water in-oil (W/O) type, milk preparations, lotions, creams, ointments, gels, serum, powders, masks, packs, sprays, aerosols, sticks, and patches. For a transdermal patch, the active agent layer includes one or more active agents, one of which is Compound I. In certain embodiments, the matrix is an adhesive matrix. The matrix may include polymeric materials. Suitable polymers for the adhesive matrix include, but are not limited to: polyurethanes, acrylates, styrenic block copolymers, silicones, and the like. For example, the adhesive matrix may include, but is not limited to, an acrylate polymer, polysiloxanes, polyisobutylene (PIB), polyisoprene, polybutadiene, styrenic block polymers, combinations of thereof, and the like. Additional examples of adhesives are described in Satas, “Acrylic Adhesives,” Handbook of Pressure-Sensitive Adhesive Technology, 2nd ed., pp. 396-456 (D. Satas, ed.), Van Nostrand Reinhold, New York (1989), the disclosure of which is herein incorporated by reference.

In certain embodiments, the active agent layer includes a permeation enhancer. The permeation enhancer may include, but is not limited to the following: aliphatic alcohols, such as but not limited to saturated or unsaturated higher alcohols having 12 to 22 carbon atoms, such as oleyl alcohol and lauryl alcohol; fatty acids, such as but not limited to linolic acid, oleic acid, linolenic acid, stearic acid, isostearic acid and palmitic acid; fatty acid esters, such as but not limited to isopropyl myristate, diisopropyl adipate, and isopropyl palmitate; alcohol amines, such as but not limited to triethanolamine, triethanolamine hydrochloride, and diisopropanolamine; polyhydric alcohol alkyl ethers, such as but not limited to alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as but not limited to polyoxyethylene alkyl ethers in which the number of carbon atoms of the alkyl group moiety is 6 to 20, and the number of repeating units (e.g. —OCH₂CH₂—) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as but not limited to glycerol esters of fatty acids having 6 to 18 carbon atoms, diglycerides, triglycerides or combinations thereof. In some embodiments, the polymer matrix includes a polyvinylpyrrolidone. The composition may further include one or more fillers or one or more antioxidants. In some embodiments, the transdermal formulations described may have a multi-layer structure. For example, the transdermal formulation may have an adhesive matrix and a backing.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

A solid form of Compound 1 of the present invention can also be administered in sustained release forms or from sustained release drug delivery systems. A description of representative sustained release materials can be found in Remington's Pharmaceutical Sciences.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Formulations for Administration, e.g., Oral Administration

Provided herein is a method for treating or preventing a disorder described herein, comprising orally administering a total daily dose of Compound 1,

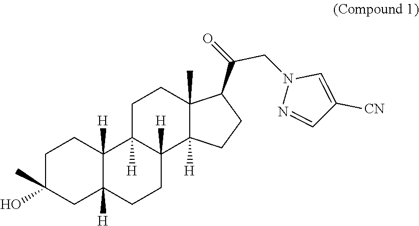

or a pharmaceutically acceptable salt or isotopologue thereof, or a pharmaceutical composition thereof of about 10 mg to about 100 mg to a subject in need thereof.

Also provided herein is a method for treating or preventing a disorder described herein, comprising orally administering a total daily dose of Compound 1, or a pharmaceutically acceptable salt or isotopologue thereof, or a pharmaceutical composition thereof of about 30 mg, e.g., 30 mg, to a subject in need thereof.

Also provided herein is a method for treating or preventing a disorder described herein, comprising orally administering a total daily dose of Compound 1 of about 30 mg, e.g., 30 mg, to a subject in need thereof.

Compound 1 or a pharmaceutically acceptable salt or isotopologue thereof, or a pharmaceutical composition thereof can be formulated for oral administration. In some embodiments of the disclosure, pharmaceutical compositions are formed into dosing forms that can be administered orally, for example by the mouth (Per os (P.O.)). Oral administration can be in the form of a tablet, capsule, chewable capsule, time-release or sustained-release tablets and capsules, and/or powders or granules. Oral administration can typically involve swallowing so that the compound enters the gastrointestinal tract (GIT). Additional dosage forms or dosing units for oral administration include solid formulations such as tablets, capsules containing particulates or powders, sachets, vials, powders, granules, lozenges, reconstitutable powders and liquid preparations (such as suspensions, emulsions and elixirs).

Oral dosage forms can contain further excipients such as binding agents (for example syrup, acacia, gelatin, sorbitol, starch, PVP, HPMC, and tragacanth); fillers (for example lactose, sugar, maize-starch, calcium phosphate, sorbitol and glycine); tabletting lubricants (for example magnesium stearate); glidants (e.g., magnesium trisilicate, powdered cellulose, starch, talc and tribasic calcium phosphate) and disintegrants (for example starch, sodium starch glycollate and microcrystalline cellulose). In addition, the oral dosage form can contain preservatives, anti-oxidant, flavours, granulation binders, wetting agents and colourants.

The amount of each type of additive employed, e.g. glidant, binder, disintegrant, filler or diluent and lubricant may vary within ranges conventional in the art. Thus for example, the amount of glidant may vary within a range of from 0.1 to 10% by weight, in particular 0.1 to 5% by weight, e.g. 0.1 to 0.5% by weight; the amount of binder may vary within a range of from about 10 to 45% by weight, e.g. 20 to 30% by weight; the amount of disintegrant may vary within a range of from 2 to 20% by weight, e.g. 15% by weight; the amount of filler or diluent may vary within a range of from 15 to 40% by weight; whereas the amount of lubricant may vary within a range of from 0.1 to 5.0% by weight.

It should be appreciated that oral dosage forms can be prepared using techniques known in the art. The absolute amounts of each additive and the amounts relative to other additives is similarly dependent on the desired properties of the solid oral dosage form and may also be chosen by the skilled artisan.

In some embodiments, Compound 1 is formulated into a solid dosage form, such as a capsule or a tablet. Capsules may be hard-shelled capsules or soft-shelled capsules. Both of these classes of capsules may be made from aqueous solutions of gelling agents, such as animal protein (mainly gelatin) or plant polysaccharides or their derivatives (such as carrageenans and modified forms of starch and cellulose). Other ingredients can be added to the gelling agent solution including plasticizers such as glycerin or sorbitol to decrease the capsule's hardness, coloring agents, preservatives, disintegrants, lubricants and surface treatment. Tablets may be defined as the solid unit dosage form of medicament or medicaments with or without suitable excipients and prepared either by molding or by compression. It may comprise a mixture of active substances and excipients, usually in powder form, pressed or compacted from a powder into a solid dose. The excipients can include diluents, binders or granulating agents, glidants (flow aids) and lubricants to ensure efficient tabletting; disintegrants to promote tablet break-up in the digestive tract; sweeteners or flavours to enhance taste; and pigments to make the tablets visually attractive or aid in visual identification of an unknown tablet. A polymer coating may be applied to make the tablet smoother and easier to swallow, to control the release rate of the active ingredient, to make it more resistant to the environment (extending its shelf life), or to enhance the tablet's appearance

In some embodiments, Compound 1 is formulated into a solid unit dose, or a solid dosage form. In some embodiments, the solid dosage form contains about 0.1 to about 10 mg of Compound 1. In some embodiments, Compound 1 is provided in a solid dosage form that contains about 5 mg to about 50 mg. In some embodiments, Compound 1 is provided in a solid dosage form that contains about 10 mg to about 100 mg. In some embodiments, Compound 1 is provided in a solid dosage form that contains about 0.5 mg, about 1 mg, about 3 mg, about 5 mg, about 10 mg, about 12 mg, about 15 mg, about 18 mg, about 20 mg, about 25 mg, about 28 mg, about 30 mg, e.g., 30 mg, about 33 mg, about 35 mg, about 40 mg, about 50 mg, about 55 mg, about 60 mg, about 65 mg, about 70 mg, about 75 mg, about 80 mg, about 85 mg, about 90 mg, about 95 mg, about 100 mg, about 105 mg, or about 110 mg of Compound 1. In some embodiments, the solid dosage form contains about 30 mg, e.g., 30 mg, of Compound 1.

### Dosage and Pharmacokinetics

The compositions described herein include a therapeutically effective amount of a neuroactive steroid, such as Compound 1, provided in a dosage form suitable for oral administration. In some embodiments, the compositions described herein include a therapeutically effective amount of a neuroactive steroid, such as Compound 1, provided in a solid dosage form suitable for oral administration. In some embodiments, the compositions described herein include a therapeutically effective amount of a neuroactive steroid, such as Compound 1, provided in a solid dosage form suitable for oral administration, which is administered substantially contemporaneously with food, as described herein.

Area under the curve (AUC) refers to the area under the curve that tracks the serum concentration (e.g., ngl/mL) of neuroactive steroid over a given time following the oral administration of the reference neuroactive steroid standard. By “reference neuroactive steroid” is intended the formulation of neuroactive steroid that serves as the basis for determination of the total hourly neuroactive steroid dose to be administered to a human subject with tremor (e.g., essential tremor), depression (e.g., postpartum depression), or an anxiety disorder to achieve the desired positive effect, i.e., a positive therapeutic response that is improved with respect to that observed without administration of neuroactive steroid. In an embodiment, the dose of neuroactive steroid to be administered provides a final serum level of neuroactive steroid of about 100 ng/mL to about 1000 ng/mL, about 1100 ng/mL to about 1450 ng/mL, 100 ng/mL to about 250 ng/mL, about 200 ng/mL to about 350 ng/mL, about 300 ng/mL to about 450 ng/mL, about 350 ng/mL to about 450 ng/mL, about 400 ng/mL to about 550 ng/mL, about 500 ng/mL to about 650 ng/mL, about 600 ng/mL to about 750 ng/mL, about 700 ng/mL to about 850 ng/mL, about 800 ng/mL to about 950 ng/mL, about 900 ng/mL to about 1050 ng/mL, about 1000 ng/mL to about 1150 ng/mL, about 100 ng/mL to about 1250 ng/mL, about 1200 ng/mL to about 1350 ng/mL, about 1300 ng/mL to about 1500 ng/m. In specific embodiments, the serum level of neuroactive steroid is about 100 ng/mL, 250 ng/mL, 300 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 500 ng/mL, 750 ng/mL, 900 ng/mL, 1200 ng/mL, 1400 ng/mL, or 1600 ng/mL.

In an embodiment, the dose of neuroactive steroid to be administered provides a final serum level of neuroactive steroid of about 100 nmoles/L to about 5000 nmoles/L, about 100 nmoles/L to about 2500 nmoles/L, about 100 nmoles/L to about 1000 nmoles/L, 100 nmoles/L to about 500 nmoles/L, about 100 nmoles/L to about 250 nmoles/L, about 100 nmoles/L to about 200 nmoles/L, about 125 nmoles/L to about 175 nmoles/L. or about 140 nmoles/L to about 160 nmoles/L. In specific embodiments, the serum level of neuroactive steroid is about 100 nmoles/L, 125 nmoles/L, 150 nmoles/L, 175 nmoles/L, 200 nmoles/L, 250 nmoles/L, 300 nmoles/L, 350 nmoles/L, 500 nmoles/L, 750 nmoles/L, 1000 nmoles/L, 1500 nmoles/L, 2000 nmoles/L, 2500 nmoles/L, or 5000 nmoles/L.

### Methods of Use

Provided herein are methods of treating a disorder, e.g., a CNS-related disorder, in a subject in need thereof, comprising administering to the subject an effective amount of Compound 1, or a pharmaceutically acceptable salt or pharmaceutically acceptable composition thereof. In certain embodiments, the disorder is a CNS-related disorder selected from the group consisting of a sleep disorder, a mood disorder, a schizophrenia spectrum disorder, a convulsive disorder, a disorder of memory and/or cognition, a movement disorder, a personality disorder, autism spectrum disorder, pain, traumatic brain injury, a vascular disease, a substance abuse disorder and/or withdrawal syndrome, and tinnitus. In some embodiments, the disorder is depression, e.g., major depressive disorder. In some embodiments, the disorder is a comorbid disorder (e.g., depression comorbid with a personality disorder or a sleep disorder comorbid with a personality disorder). In some embodiments, the disorder is a neurological disorder as described herein. In some embodiments, the disorder is a neurological disorder as described herein. In some embodiments, the disorder is a psychiatric disorder as described herein. In some embodiments, the disorder is a seizure disorder as described herein. In some embodiments, the disorder is a neuroinflammatory disorder as described herein. In some embodiments, the disorder is a glaucoma or metabolic disorder as described herein. In some embodiments, the disorder is a sensory deficit disorder as described herein. Also provided herein are methods of using Compound 1, or a pharmaceutically acceptable salt or pharmaceutical composition thereof, as a neuroprotectant. Also provided herein are methods of using Compound 1, or a pharmaceutically acceptable salt or pharmaceutical composition thereof, as an analgesic or other agent for pain control.

### Neurological Disorders

Compound 1, or a pharmaceutically acceptable salt or pharmaceutically acceptable composition thereof, can be used in a method described herein, for example in the treatment of a disorder described herein such as a neurological disorder. Exemplary neurological disorders include, but are not limited to, neurodegenerative disorders, neurodevelopmental disorders, neuroendocrine disorders and dysfunction, movement disorders, and sleep disorders as described herein.

### Neurodegenerative Disorders

Compound 1, or a pharmaceutically acceptable salt or pharmaceutically acceptable composition thereof, can be used in a method described herein, for example in the treatment of a neurodegenerative disorder.

The term “neurodegenerative disease” includes diseases and disorders that are associated with the progressive loss of structure or function of neurons, or death of neurons. Neurodegenerative diseases and disorders include, but are not limited to, Alzheimer's disease (including the associated symptoms of mild, moderate, or severe cognitive impairment); amyotrophic lateral sclerosis (ALS); anoxic and ischemic injuries; benign forgetfulness; brain edema; cerebellar ataxia including McLeod neuroacanthocytosis syndrome (MLS); closed head injury; coma; contusive injuries (e.g., spinal cord injury and head injury); dementias including multi-infarct dementia and senile dementia; disturbances of consciousness; Down syndrome; fragile X syndrome; Gilles de la Tourette's syndrome; head trauma; hearing impairment and loss; Huntington's disease; Lennox syndrome; mental retardation; neuronal damage including ocular damage, retinopathy or macular degeneration of the eye; neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest; Parkinson's disease; stroke; tinnitus; tubular sclerosis, and viral infection induced neurodegeneration (e.g., caused by acquired immunodeficiency syndrome (AIDS) and encephalopathies). Neurodegenerative diseases also include, but are not limited to, neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. Methods of treating or preventing a neurodegenerative disease also include treating or preventing loss of neuronal function characteristic of neurodegenerative disorder.

### Neurodevelopmental Disorders

Compound 1, or a pharmaceutically acceptable salt or pharmaceutically acceptable composition thereof, can be used in a method described herein, for example in the treatment of a disorder described herein such as a neurodevelopmental disorder. In some embodiments, the neurodevelopmental disorders are autism spectrum disorder. In some embodiments, the neurodevelopmental disorder is Smith-Lemli-Opitz syndrome.

### Neuroendocrine Disorders

Provided herein are methods that can be used for treating neuroendocrine disorders and dysfunction. As used herein, “neuroendocrine disorder” or “neuroendocrine dysfunction” refers to a variety of conditions caused by imbalances in the body's hormone production directly related to the brain. Neuroendocrine disorders involve interactions between the nervous system and the endocrine system. Because the hypothalamus and the pituitary gland are two areas of the brain that regulate the production of hormones, damage to the hypothalamus or pituitary gland, e.g., by traumatic brain injury, may impact the production of hormones and other neuroendocrine functions of the brain. In some embodiments, the neuroendocrine disorder or dysfunction is associated with a women's health disorder or condition (e.g., a women's health disorder or condition described herein). In some embodiments, the neuroendocrine disorder or dysfunction is associated with a women's health disorder or condition is polycystic ovary syndrome.

Symptoms of neuroendocrine disorder include, but are not limited to, behavioral, emotional, and sleep-related symptoms, symptoms related to reproductive function, and somatic symptoms; including but not limited to fatigue, poor memory, anxiety, depression, weight gain or loss, emotional lability, lack of concentration, attention difficulties, loss of libido, infertility, amenorrhea, loss of muscle mass, increased belly body fat, low blood pressure, reduced heart rate, hair loss, anemia, constipation, cold intolerance, and dry skin.

### Movement Disorders

Compound 1, or a pharmaceutically acceptable salt or pharmaceutically acceptable composition thereof, can be used in a method described herein, for example in the treatment of a movement disorder. In some embodiments, the movement disorder is essential Tremor, Stiff-Person syndrome, spasticity, Freidrich's ataxia, Cerebellar ataxia, dystonia, Tourette Syndrome, Fragile X-associated tremor or ataxia syndromes, drug-induced or medication-induced Parkinsonism (such as neuroleptic-induced acute akathisia, acute dystonia, Parkinsonism, or tardive dyskinesia, neuroleptic malignant syndrome, or medication-induced postural tremor), ataxia, cerebellar ataxia including McLeod neuroacanthocytosis syndrome (MLS), levodopa-induced dyskinesia, movement disorders including akinesias and akinetic (rigid) syndromes (including basal ganglia calcification, corticobasal degeneration, multiple system atrophy, Parkinsonism-ALS dementia complex, Parkinson's disease, postencephalitic parkinsonism, and progressively supranuclear palsy); muscular spasms and disorders associated with muscular spasticity or weakness including chorea (such as benign hereditary chorea, drug-induced chorea, hemiballism, Huntington's disease, neuroacanthocytosis, Sydenham's chorea, and symptomatic chorea), dyskinesia (including tics such as complex tics, simple tics, and symptomatic tics), myoclonus (including generalized myoclonus and focal cyloclonus), tremor (such as rest tremor, postural tremor, and intention tremor), or dystonia (including axial dystonia, dystonic writer's cramp, hemiplegic dystonia, paroxysmal dystonia, and focal dystonia such as blepharospasm, oromandibular dystonia, and spasmodic dysphonia and torticollis).

As used herein, “movement disorders” refers to a variety of diseases and disorders that are associated with hyperkinetic movement disorders and related abnormalities in muscle control. Exemplary movement disorders include, but are not limited to, Parkinson's disease and parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders. Exemplary movement disorders include, but are not limited to, Parkinson's disease and parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

### Tremor

The methods described herein can be used to treat tremor, for example, Compound 1, or a pharmaceutically acceptable salt or pharmaceutically acceptable composition thereof, can be used to treat cerebellar tremor or intention tremor, dystonic tremor, essential tremor, orthostatic tremor, parkinsonian tremor, physiological tremor, psychogenic tremor, or rubral tremor. Tremor includes hereditary, degenerative, and idiopathic disorders such as Wilson's disease, Parkinson's disease, and essential tremor, respectively; metabolic diseases (e.g., thyoid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor.

Tremor is an involuntary, at times rhythmic, muscle contraction and relaxation that can involve oscillations or twitching of one or more body parts (e.g., hands, arms, eyes, face, head, vocal folds, trunk, and legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawl, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's Disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's Disease, but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face. Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myloclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example simple motor tics involve only a few muscles restricted to a specific body part.

Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and includes hemiplegic gait, diplegic gait, neuropathic gait, myopathic gait, parkinsonian gait, choreiform gait, ataxic gait, and sensory gait.

### Sleep Disorders

Compound 1, or a pharmaceutically acceptable salt or pharmaceutically acceptable composition thereof, can be used in a method described herein, for example in the treatment of a sleep disorder. In some embodiments, the sleep disorder is comorbid with another disorder (e.g., a sleep disorder comorbid with a personality disorder).

### Psychiatric Disorders

Compound 1, or a pharmaceutically acceptable salt or pharmaceutically acceptable composition thereof, can be used in a method described herein, for example in the treatment of a disorder described herein such as a psychiatric disorder. Exemplary psychiatric disorders include, but are not limited to, mood disorders, anxiety disorders, psychotic disorders, and impulse control disorders as described herein.

### Mood Disorders

Also provided herein are methods for treating a mood disorder, for example, clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, comorbid depression, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression or a symptom of depression during pregnancy. This depression is referred to herein as perinatal depression. In an embodiment, a subject experiencing perinatal depression is at increased risk of experiencing PND. Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporous, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years). Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Depression or personality disorders may also be comorbid with another disorder. For example, depression may be comorbid with a personality disorder. In another example, a personality disorder may be comorbid with a sleep disorder.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

### Anxiety Disorders

Provided herein are methods for treating anxiety disorders (e.g., generalized anxiety disorder, panic disorder, obsessive compulsive disorder, phobia, post-traumatic stress disorder). Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Psychotic Disorders

Compound 1, or a pharmaceutically acceptable salt or pharmaceutically acceptable composition thereof, can be used in a method described herein, for example in the treatment of a psychotic disorder. In some embodiments, the impulse control disorder is schizophrenia or bipolar disorder. In some embodiments, the psychotic disorder is schizophrenia. In some embodiments, the psychotic disorder is bipolar disorder.

Bipolar disorder or manic depressive disorder causes extreme mood swings that include emotional highs (mania or hypomania) and lows (depression).

### Impulse Control Disorders

Compound 1, or a pharmaceutically acceptable salt or pharmaceutically acceptable composition thereof, can be used in a method described herein, for example in the treatment of an impulse control disorder. In some embodiments, the impulse control disorder is anorexia nervosa or alcohol withdrawal. In some embodiments, the impulse control disorder is anorexia nervosa. In some embodiments, the impulse control disorder is anorexia nervosa.

### Seizure Disorders

Compound 1, or a pharmaceutically acceptable salt or pharmaceutically acceptable composition thereof, can be used in a method described herein, for example in the treatment of a seizure disorder. In some embodiments, the seizure disorder is epilepsy. In some embodiments, the seizure disorder is status epilepticus, e.g., convulsive status epilepticus, e.g., early status epilepticus, established status epilepticus, refractory status epilepticus, or super-refractory status epilepticus. In some embodiments, the seizure disorder is a focal seizure with either motor (automatisms, atonic, clonic, epileptic spasms, hyperkinetic, myoclonic, and tonic) or non-motor (autonomic, behavioral arrest, cognition, emotional, and sensory) onset, a generalized seizure with either motor (tonic-clonic, clonic, myoclonic, myoclonic-tonic-clonic, myoclonic-atonic, atonic, epileptic spasms) or non-motor (absence) onset, a seizure with unknown motor (tonic-clonic, epileptic spasms) or non-motor (behavioral arrest) onset, a seizure associated with clinical syndromes, such as Dravet syndrome, Rett syndrome, Lennox Gasteau syndrome, Tuberous sclerosis, Angelmans syndrome, catamenial epilepsy. In some embodiments, the seizure disorder is a seizure that is caused by schizoaffective disorder or by drugs used to treat schizophrenia.

### Epilepsy

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Status Epilepticus (SE)

Status epilepticus (SE) can include, e.g., convulsive status epilepticus, e.g., early status epilepticus, established status epilepticus, refractory status epilepticus, or super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus. There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Neuroinflammatory Disorders

Compound 1, or a pharmaceutically acceptable salt or pharmaceutically acceptable composition thereof, can be used in a method described herein, for example in the treatment of a disorder described herein such as a neuroinflammatory disorder. In some embodiments, the neuroinflammatory disorder is multiple sclerosis or a pediatric autoimmune neuropsychiatric disorder associated with a streptococcal infection (PANDAS).

### Analgesia/Pain Control

Compound 1, or a pharmaceutically acceptable salt or pharmaceutically acceptable composition thereof, can be used in a method described herein, for example as an analgesic or other agent for pain control. In some embodiments, a solid form of Compound 1, or a pharmaceutically acceptable composition thereof, can be used as an analgesic or other agent for pain control to treat inflammatory pain, neuropathic pain, fibromyalgia, or peripheral neuropathy.

### Sensory Deficit Disorders

Compound 1, or a pharmaceutically acceptable salt or pharmaceutically acceptable composition thereof, can be used in a method described herein, for example in the treatment of a disorder described herein such as a sensory deficit disorder. In some embodiments, the sensory deficit disorder is tinnitus or synesthesia. In some embodiments, the sensory deficit disorder is hearing impairment and/or loss.

### Methods of Evaluating Treatment for Disorders, e.g., Major Depressive Disorder

Also provided herein are methods for evaluating subjects with a depressive disorder, e.g., major depressive disorder, before and after receiving a treatment with a therapeutic, e.g., Compound 1. In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. Reduction in the HAM-D total score can happen within 4, 3, 2, or 1 days; or 96, 84, 72, 60, 48, 24, 20, 16, 12, 10, 8 hours or less. The therapeutic effect can be assessed across a specified treatment period. For example, the therapeutic effect can be determined by a decrease from baseline in HAM-D total score after administering Compound 1 (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with Compound 1) is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with Compound 1 is greater than or equal to 23. In some embodiments, the baseline score is at least 10, 15, or 20. In some embodiments, the HAM-D total score of the subject after treatment with Compound 1 is about 0 to 10 (e.g., less than 10; 0 to 10, 0 to 6, 0 to 4, 0 to 3, 0 to 2, or 1.8). In some embodiments, the HAM-D total score after treatment with Compound 1 is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with Compound 1. In some embodiments, the decrease in the baseline HAM-D total score to HAM-D total score after treatment with Compound 1 is at least 1, 2, 3, 4, 5, 7, 10, 25, 40, 50, or 100 fold). In some embodiments, the percentage decrease in the baseline HAM-D total score to HAM-D total score after treatment with Compound 1 is at least 50% (e.g., 60%, 70%, 80%, or 90%). In some embodiments, the therapeutic effect is measured as a decrease in the HAM-D total score after treatment with Compound 1 relative to the baseline HAM-D total score (e.g., 12, 24, 48 hours after administration; or 24, 48, 72, 96 hours or more; or 1 day, 2 days, 14 days, or more) is at least 10, 15, or 20 points.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with Compound 1. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with Compound 1. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with Compound 1. In some embodiments, the therapeutic effect is a decrease from baseline in HAM-D total score after treatment with Compound 1 (e.g., treatment with Compound 1 once a day for 14 days). In some embodiments, the HAM-D total score of the subject before treatment with Compound 1 is at least 24. In some embodiments, the HAM-D total score of the subject before treatment with Compound 1 is at least 18. In some embodiments, the HAM-D total score of the subject before treatment with Compound 1 is between and including 14 and 18. In some embodiments, the decrease in HAM-D total score after treating the subject with Compound 1 relative to the baseline HAM-D total score is at least 10. In some embodiments, the decrease in HAM-D total score after treating the subject with Compound 1 relative to the baseline HAM-D total score is at least 15 (e.g., at least 17). In some embodiments, the HAM-D total score associated with treating the subject with Compound 1 is no more than a number ranging from 6 to 8. In some embodiments, the HAM-D total score associated with treating the subject with Compound 1 is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the method provides therapeutic effect (e.g., as measured by reduction in Montgomery-Asberg Depression Rating Scale (MADRS)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

In some embodiments of any of the foregoing, the subject is administered Compound 1, e.g., about a 30 mg dose of Compound 1, e.g., a 30 mg dose of Compound 1, once a day for less than 2 weeks. In some embodiments of any of the foregoing, the subject is administered Compound 1, e.g., about a 30 mg dose of Compound 1, e.g., a 30 mg dose of Compound 1, once a day for 1 day. In some embodiments of any of the foregoing, the subject is administered Compound 1, e.g., about a 30 mg dose of Compound 1, e.g., a 30 mg dose of Compound 1, once a day for 2 days. In some embodiments of any of the foregoing, the subject is administered Compound 1, e.g., about a 30 mg dose of Compound 1, e.g., a 30 mg dose of Compound 1, once a day for at least 14 days. In some embodiments, the subject is administered Compound 1, e.g., about a 30 mg dose, e.g., a 30 mg dose once a day for at least 28 days. In some embodiments, the subject is administered Compound 1, e.g., about a 30 mg dose, e.g., a 30 mg dose once a day for at least 6 months. In some embodiments, the subject is administered Compound 1, e.g., about a 30 mg dose, e.g., a 30 mg dose once a day for at least 1 year. In some embodiments, the subject is administered Compound 1, e.g., about a 30 mg dose, e.g., a 30 mg dose once a day for life. In some embodiments, the subject is administered Compound 1 at night. In some embodiments, the subject is administered Compound 1 no longer than 1 hour before the subject sleeps. In some embodiments, the subject is administered Compound 1 no longer than 15 minutes before the subject sleeps. In some embodiments, Compound 1 is administered chronically.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

### Example 1

Compound 1 was assessed for safety and tolerability in Compound 1 capsules dosed in healthy subjects. Compound 1 was also assessed for the relative bioavailability of Compound 1 capsules compared to Compound 1 Oral Solutions.

In a Phase 1, single-center, open-label, four-period, two-sequence crossover study, Compound 1 capsules were evaluated for safety, tolerability, and relative bioavailability. Twelve (12) subjects completed all four periods of the study; subjects who replaced discontinued subjects were allocated to the same randomization sequence as those discontinued. Up to 24 subjects were recruited into the study.

This study consisted of four periods:

Period 1: Subjects (N=20) were randomized on a 1:1 basis to receive a single 30-mg dose of Compound 1 Capsules or a single 30-mg dose of Compound 1 Oral Solution on Day 1. Study drug was administered in the fasting state. Subjects were confined to the inpatient facility from Day −1 until they were discharged on Day 3.

Period 2: After a washout period (which ends on Day 7), the same subjects (N=20) as Period 1 were crossed over to the dose form that they did not receive in Period 1. On Day 8, study drug was administered in the fasting state. Subjects were confined to the inpatient facility from Day 7 until they were discharged on Day 10.

Period 3: Food Effect (high fat): All subjects received a single 30 mg dose of Compound 1 Capsules on Day 15. Study drug was administered after a high-fat meal. Subjects were confined to the inpatient facility from Day 14 until they were discharged on Day 17.

Period 4: Food Effect (standard): All subjects received a single 30 mg dose of Compound 1 Capsules on Day 22. Study drug was administered after a standard meal. Subjects were confined to the inpatient facility from Day 21 until they were discharged on Day 24.

Subjects were admitted on the day before the dose was scheduled to be administered (i.e., on Day −1, Day 7, Day 14 and Day 21) in order to undergo the predose assessments. Subjects had an End-of-Treatment visit on Day 28 and a follow-up evaluation 13 days after the last dose (Day 35).

The Compound 1 Capsules dose was administered as two capsules with 8 ounces (240 mL) of water; both capsules were to be swallowed as quickly as possible. Compound 1 Oral Solution was prepared as approximately 40 mL, to be swallowed all at once, followed by approximately 200 mL of water that was used to rinse the dosing bottle approximately five times. The time of swallowing the initial 40 mL solution or capsules was the time zero for all assessments.

Subjects were not allowed to consume water for 1 hour before and after dosing, except for water consumed during dosing. After 1 hour postdose, water was allowed ad libitum. Subjects were required to fast overnight (minimum of 10 hours) prior to the scheduled dosing. In periods 1 and 2, subjects were administered a standard meal 4 or more hours after the dose was administered.

In period 3 (food effect high-fat meal), subjects were administered a high-fat meal 30 minutes prior to administration of the Compound 1 Capsules. Subjects should have eaten this meal in 30 minutes or less, and the Compound 1 dose was administered 30 minutes after the start of the meal. No other food was allowed for at least 4 hours postdose.

In period 4 (food effect standard meal), subjects were administered a standard meal 30 minutes prior to administration of the Compound 1 Capsules. Subjects should have eaten this meal in 30 minutes or less, and the Compound 1 dose was administered 30 minutes after start of the meal. No other food was allowed for at least 4 hours postdose.

Plasma samples were collected for PK analysis of Compound 1 at the following sampling times relative to dosing in each period: predose on Day 1 and at 15 and 30 minutes and 1, 1.5, 2, 3, 4, 6, 8, 10, 12, 16, 24 (Day 2), and 48 (Day 3) hours postdose.

The plasma samples were kept frozen at approximately −70 to −80° C. until analyzed. They were packed as directed to avoid breakage during transit and with sufficient dry ice to prevent thawing for at least 72 hours. A specimen-identification form or equivalent was completed and sent to the laboratory with each set of samples.

Bioanalyses of plasma samples for the determination of Compound 1 were conducted utilizing validated LC-MS/MS methods at Agilux Laboratories, Worcester, Mass.

For Compound 1, the following PK parameters were calculated, as appropriate, from the individual plasma concentrations, using Phoenix® WinNonLin®6.3 or higher following these guidelines:
Actual sampling times relative to dosing rather than nominal times were used in the calculation of all derived PK parameters
There was no imputation of missing data
Any subjects with missing concentration data were included in the PK analysis set provided that at least Cₘₐₓ and AUC₀₋ₜ were reliably calculated.
At least 3 time points with measurable plasma concentrations were required for the calculation of AUC₀₋ₜ and at least 3 time points with measurable plasma concentrations after tₘₐₓ were required for the calculation of λz.
Sample values below the limit of quantification (BLQ) values in the absorption phase, before the first reported concentration, were substituted by zeros. The BLQ values between evaluable concentrations and the terminal BLQ values were set to ‘missing’. These measures were taken to prevent an over-estimation of AUC₀₋ₜ and AUC_{0-inf}
Post-dose values of ‘no sample received’ (NS), ‘insufficient sample for analysis’ (IS), or ‘not reported’ (NR) were omitted from the PK analysis. If NS, IS, or NR was reported for time points close to the estimated tₘₐₓ, the subject may have been non-evaluable for the PK purpose. If NS, IS, or NR was reported for more than two sequential time points, the subject may have been considered non-evaluable for PK purposes. Table 6 provides the definitions and units for the pharmacokinetic parameters.

TABLE 6
Pharmacokinetic Parameters

| Parameter | Unit | Definition |
|---|---|---|
| AUC_{0-inf} | ng · h/mL | AUC from the time of dosing extrapolated to |
| | | infinity |
| AUC₀₋ₜ | ng · h/mL | AUC from the time of dosing to the last |
| | | quantifiable concentration |
| Cₘₐₓ | ng/mL | Maximum observed plasma drug concentration |
| tₘₐₓ | h | Time of maximum observed concentration |
| t_{1/2} | h | Apparent terminal elimination half-life |

h, hour; mL, milliliter; ng, nanogram

A linear mixed model applied to the Compound 1 parameters AUC_{0-inf}, AUC₀₋ₜ, and Cₘₐₓ, with fixed effects terms for dosing condition (fasted solution, fasted capsules, fed capsules [high-fat meal], fed capsules [standard meal]) were used to test the effect of condition on the rate and extent of absorption. Pharmacokinetic parameters were natural log-transformed prior to analysis. An unstructured covariance matrix was used to allow for unequal dosing condition variances and to model the correlation between the dosing condition measurements within each subject via the REPEATED statement in SAS PROC MIXED. Kenward and Roger's method was used to calculate the denominator degrees of freedom for the fixed effects (DDFM=KR).

For AUC and Cₘₐₓ, the treatment ratio ‘test/reference’ was calculated by taking the anti-logarithm of the difference between treatment means. In this analysis, the Compound 1 fasted solution condition was considered the reference and the Compound 1 fasted capsules condition was the test treatment. A 90% confidence interval was constructed for the geometric mean test-to-reference ratio for both AUC and Cₘₐₓ. Evidence of an absence of dosing condition effect was concluded if the 90% confidence intervals for AUC and Cₘₐₓ were contained within the interval (0.80, 1.25). For food-effect analyses, the fed capsules conditions (standard meal, high-fat meal) was considered the test treatment and the fasted capsules condition served as the reference.

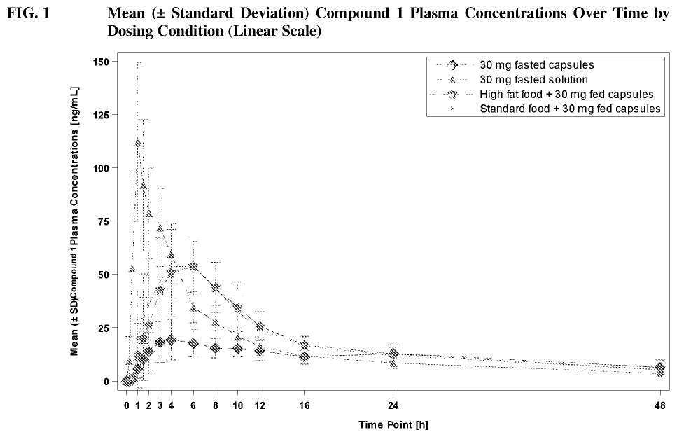

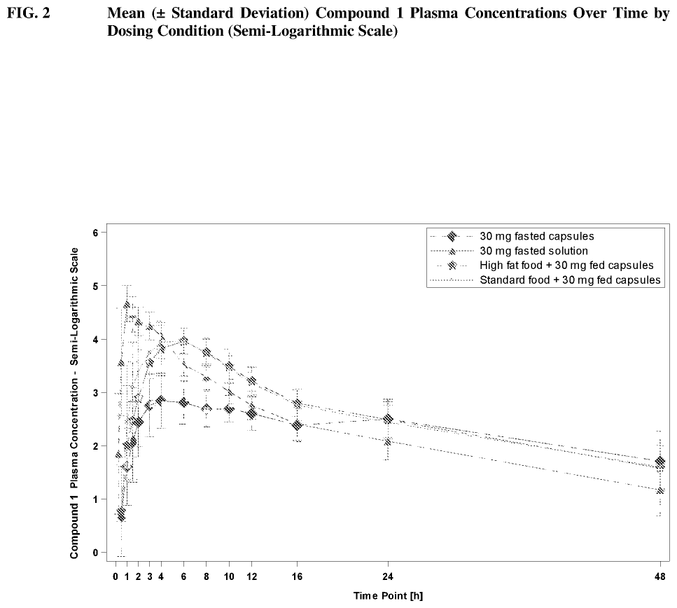

Mean (±SD) Compound 1 plasma concentration-time curves are displayed in FIG. 1 (linear scale) and FIG. 2 (semi-log scale).

Following a single oral dose of Compound 1 solution in the fasted state, the arithmetic mean Cₘₐₓ (CV %) was 119.77 ng/mL (32.087%). The median tₘₐₓ (min-max) was 1.00 hour (0.50 hour-4.00 hours). The arithmetic mean AUC₀₋ₜ (CV %) and AUC_{0-inf} (CV %) were 795.4 h*ng/mL (22.42%) and 798.4 h*ng/mL (28.60%), respectively. The arithmetic mean t_{1/2} (CV %) was 13.50 hours (21.713%).

Following a single oral dose of Compound 1 capsules in the fasted state, the arithmetic mean Cₘₐₓ (CV %) was 22.98 ng/mL (35.653%). The median tₘₐₓ (min-max) was 4.02 hour (1.50 hour-24.28 hours). The arithmetic mean AUC₀₋ₜ (CV %) and AUC_{0-inf}(CV %) were 543.7 h*ng/mL (22.03%) and 642.1 h*ng/mL (9.60%), respectively. The arithmetic mean t_{1/2} (CV %) was 15.60 hours (11.939%).

Following a single oral dose of Compound 1 capsules concurrent with a high-fat meal, the arithmetic mean Cₘₐₓ (CV %) was 64.37 ng/mL (21.814%). The median tₘₐₓ (min-max) was 5.97 hour (2.00 hour-10.00 hours). The arithmetic mean AUC₀₋ₜ (CV %) and AUC_{0-inf} (CV %) were 846.5 h*ng/mL (17.51%) and 895.7 h*ng/mL (29.16%), respectively. The arithmetic mean t_{1/2} (CV %) was 12.94 hours (12.507%).

Following a single oral dose of Compound 1 capsules concurrent with a standard meal, the arithmetic mean Cₘₐₓ (CV %) was 65.16 ng/mL (25.715%). The median tₘₐₓ (min-max) was 4.00 hour (1.00 hour-8.00 hours). The arithmetic mean AUC₀₋ₜ (CV %) and AUC_{0-inf} (CV %) were 851.8 h*ng/mL (18.03%) and 1020 h*ng/mL (12.92%), respectively. The arithmetic mean t_{1/2} (CV %) was 14.90 hours (16.115%).

The above results are summarized in Table 8:

TABLE 8
Summary of Compound 1 Plasma Pharmacokinetic
Parameters (Pharmacokinetic Analysis Set)

| Dosing | | Cₘₐₓ | tₘₐₓ | t_{1/2} | AUC₀₋ₜ | AUC_{0-inf} |
|---|---|---|---|---|---|---|
| Condition | Statistic | (ng/mL) | (h) | (h) | (h*ng/mL) | (h*ng/mL) |
| Fasted | n | 21 | 21 | 5 | 21 | 5 |
| Solution | Mean | 119.77 | | 13.50 | 795.4 | 798.4 |
| | SD | 38.430 | | 2.932 | 178.4 | 228.3 |
| | Median | 115.00 | 1.00 | 13.27 | 788.2 | 816.4 |
| | Min | 65.9 | 0.50 | 10.35 | 476.4 | 500.9 |
| | Max | 208.0 | 4.00 | 18.19 | 1167 | 1046 |
| | % CV | 32.087 | | 21.713 | 22.42 | 28.60 |
| | G. Mean | 114.3 | | | 776.3 | 770.3 |
| | G. % CV | 31.924 | | | 23.14 | 31.32 |
| Fasted | n | 20 | 20 | 5 | 20 | 5 |
| Capsules | Mean | 22.98 | | 15.60 | 543.7 | 642.1 |
| | SD | 8.191 | | 1.862 | 119.8 | 61.67 |
| | Median | 20.40 | 4.02 | 15.72 | 551.9 | 650.1 |
| | Min | 13.0 | 1.50 | 12.87 | 273.3 | 572.8 |
| | Max | 42.8 | 24.28 | 17.67 | 744.9 | 715.9 |
| | % CV | 35.653 | | 11.939 | 22.03 | 9.60 |
| | G. Mean | 21.8 | | | 529.6 | 639.7 |
| | G. % CV | 32.834 | | | 24.79 | 9.67 |
| Fed | n | 19 | 19 | 4 | 19 | 4 |
| Capsules | Mean | 64.37 | | 12.94 | 846.5 | 895.7 |
| [high-fat | SD | 14.041 | | 1.618 | 148.3 | 261.2 |
| meal] | Median | 66.30 | 5.97 | 12.70 | 822.0 | 877.6 |
| | Min | 35.6 | 2.00 | 11.54 | 552.4 | 595.1 |
| | Max | 90.3 | 10.00 | 14.80 | 1159 | 1232 |
| | % CV | 21.814 | | 12.507 | 17.51 | 29.16 |
| | G. Mean | 62.8 | | | 834.1 | 866.9 |
| | G. % CV | 23.755 | | | 18.00 | 30.44 |
| Fed | n | 19 | 19 | 5 | 19 | 5 |
| Capsules | Mean | 65.16 | | 14.90 | 851.8 | 1020 |
| [standard | SD | 16.757 | | 2.401 | 153.6 | 131.8 |
| meal] | Median | 63.20 | 4.00 | 15.84 | 880.8 | 1061 |
| | Min | 34.5 | 1.00 | 10.81 | 563.1 | 828.5 |
| | Max | 108.0 | 8.00 | 16.77 | 1050 | 1144 |
| | % CV | 25.715 | | 16.115 | 18.03 | 12.92 |
| | G. Mean | 63.1 | | | 837.6 | 1013 |
| | G. % CV | 26.806 | | | 19.54 | 13.51 |

AUC = Area under the curse;
Cₘₐₓ = Maximum concentration;
G. = geometric;
tₘₐₓ = Time of maximum concentration;
t_{1/2} = half-life
*Summarized by Median, minimum and maximum

The statistical analysis of the plasma pharmacokinetics of Compound 1 when administered as a solution in the fasted state as compared with a capsule in the fasted state (Table 9) indicates a decreased Cₘₐₓ for the capsule; the geometric mean ratio (90% CI) was 0.191 (0.17, 0.22). The Compound 1 AUC₀₋ₜ was also decreased; the geometric mean ratio (90% CI) was 0.682 (0.62, 0.75). There were an inadequate number of subjects with profiles accurately capturing AUC_{0-inf}, so only a geometric mean ratio was presented (0.830).

The statistical analysis of the plasma pharmacokinetics of Compound 1 when administered as capsules in the fed state (high-fat meal) as compared with capsules in the fasted state (Table 10) indicates an increased Cₘₐₓ for the fed state; the geometric mean ratio (90% CI) was 2.879 (2.56, 3.28). The Compound 1 AUC₀₋ₜ was also increased; the geometric mean ratio (90% CI) was 1.575 (1.45, 1.69). There were an inadequate number of subjects with profiles accurately capturing AUC_{0-inf}, so only a geometric mean ratio is presented (1.355).

The statistical analysis of the plasma pharmacokinetics of Compound 1 when administered as capsules in the fed state (standard meal) as compared with capsules in the fasted state (Table 11) indicates an increased Cₘₐₓ for the fed state; the geometric mean ratio (90% CI) was 2.894 (2.64, 3.25). The Compound 1 AUC₀₋ₜ was also increased; the geometric mean ratio (90% CI) was 1.581 (1.43, 1.72). There were an inadequate number of subjects with profiles accurately capturing AUC_{0-inf}, so only a geometric mean ratio is presented (1.584).

The statistical analysis of the plasma pharmacokinetics of Compound 1 when administered as capsules in the fed state (high-fat meal) as compared with a solution in the fasted state (Table 12) indicates an decreased Cₘₐₓ for the fed state; the geometric mean ratio (90% CI) was 0.549 (0.48, 0.63). The Compound 1 AUC₀₋ₜ values were comparable; the geometric mean ratio (90% CI) was 1.074 (1.02, 1.12). There were an inadequate number of subjects with profiles accurately capturing AUC_{0-inf}, so only a geometric mean ratio is presented (1.125).

The statistical analysis of the plasma pharmacokinetics of Compound 1 when administered as capsules in the fed state (standard meal) as compared with a solution in the fasted state (Table 13) indicates a decreased Cₘₐₓ for the fed state; the geometric mean ratio (90% CI) was 0.552 (0.49, 0.64). The Compound 1 AUC₀₋ₜ values were comparable; the geometric mean ratio (90% CI) was 1.079 (1.02, 1.13). There were an inadequate number of subjects with profiles accurately capturing AUC_{0-inf}, so only a geometric mean ratio is presented (1.315).

TABLE 9
Statistical Analysis of the Effect of Dosing Condition on Compound
1 Plasma Pharmacokinetic Parameters (Pharmacokinetic Analysis Set)

| | Fasted Capsules | | | Fasted Solution | | | Test/Reference | |
|---|---|---|---|---|---|---|---|---|
| Parameter | N[1] | GM | 95% CI | N[1] | GM | 95% CI | GMR | 90% CI |
| Cₘₐₓ | 20 | 21.81 | (18.81, 25.34) | 21 | 114.30 | (99.19, 131.72) | 0.191 | (0.17, 0.22) |
| (ng/mL) | | | | | | | | |
| AUC₀₋ₜ | 20 | 529.62 | (474.24, 594.73) | 21 | 776.27 | (699.62, 861.31) | 0.682 | (0.62, 0.75) |
| (h*ng/mL) | | | | | | | | |
| AUC_{0-inf} | 5 | 639.69 | * | 5 | 770.31 | * | 0.830 | * |
| (h*ng/mL) | | | | | | | | |

In this analysis, a linear mixed model applied to the Compound 1 PK parameters AUC_{0-inf}, AUC₀₋ₜ, and Cₘₐₓ, with fixed effects terms fasted status will be used to test the effect of dosing form (solution and capsules) on the rate and extent of absorption. An unstructured covariance matrix will be used. The Compound 1 fasted solution condition will be considered the reference and the Compound 1 fasted capsules condition will be the test treatment. Evidence of an absence of dosing condition effect will be concluded if the 90% CI for AUC and Cₘₐₓ are contained within the interval (0.8, 1.25).
[1]Shows the number of subjects exposed to each treatment condition that were used in the mixed model.
GM = Geometric means, GMR = Ratio of Geometric Means
*: Non-convergence caused by sparse data for AUC_{0-inf}

TABLE 10
Statistical Analysis of the Effect of High-fat Meal for Compound 1
Plasma Pharmacokinetic Parameters (Pharmacokinetic Analysis Set)

| | Fed Capsules | | | | | | | |
|---|---|---|---|---|---|---|---|---|
| | [high-fat meal] | | | Fasted Capsules | | | Test/Reference | |
| Cₘₐₓ | 19 | 62.80 | (56.49, 70.87) | 20 | 21.81 | (18.81, 25.34) | 2.879 | (2.56, 3.28) |
| (ng/mL) | | | | | | | | |
| AUC₀₋ₜ | 19 | 834.06 | (766.99, 901.60) | 20 | 529.62 | (474.24, 594.73) | 1.575 | (1.45, 1.69) |
| (h*ng/mL) | | | | | | | | |
| AUC_{0-inf} | 4 | 866.90 | * | 5 | 639.69 | * | 1.355 | * |
| (h*ng/mL) | | | | | | | | |

In this analysis, a linear mixed model applied to the Compound 1 PK parameters AUC_{0-inf}, AUC₀₋ₜ, and Cₘₐₓ, with fixed effects terms dosing form (capsules) will be used to test the effect of high-fat meal on the rate and extent of absorption. An unstructured covariance matrix will be used. The Compound 1 fasted capsules condition will be considered the reference and the Compound 1 fed capsules [high-fat meal] will be the test treatment. Evidence of an absence of high-fat meal effect will be concluded if the 90% CI for AUC and Cₘₐₓ are contained within the interval (0.8, 1.25).
[1] Shows the number of subjects exposed to each treatment condition that were used in the mixed model.
GM = Geometric means, GMR = Ratio of Geometric Means
*: Non-convergence caused by sparse data for AUC_{0-inf}

TABLE 11
Statistical Analysis of the Effect of Standard Meal for Compound 1
Plasma Pharmacokinetic Parameters (Pharmacokinetic Analysis Set)

| | Fed Capsules | | | | | | | |
|---|---|---|---|---|---|---|---|---|
| | [Standard meal] | | | Fasted Capsules | | | Test/Reference | |
| Cₘₐₓ | 19 | 63.12 | (56.34, 2.62) | 20 | 21.81 | (18.81, 5.34) | 2.894 | (2.64, 3.25) |
| (ng/mL) | | | | | | | | |
| AUC₀₋ₜ | 19 | 837.55 | (763.47, 911.27) | 20 | 529.62 | (474.24, 594.73) | 1.581 | (1.43, 1.72) |
| (h*ng/mL) | | | | | | | | |
| AUC_{0-inf} | 5 | 1013.04 | * | 5 | 639.69 | * | 1.584 | * |
| (h*ng/mL) | | | | | | | | |

In this analysis, a linear mixed model applied to the Compound 1 PK parameters AUC_{0-inf}, AUC₀₋ₜ, and Cₘₐₓ, with fixed effects terms dosing form (capsules) will be used to test the effect of standard-fat meal on the rate and extent of absorption. An unstructured covariance matrix will be used. The Compound 1 fasted capsules condition will be considered the reference and the Compound 1 fed capsules [standard-fat meal] will be the test treatment. Evidence of an absence of standard-fat meal effect will be concluded if the 90% CI for AUC and Cₘₐₓ are contained within the interval (0.8, 1.25).
[1] Shows the number of subjects exposed to each treatment condition that were used in the mixed model.
GM = Geometric means, GMR = Ratio of Geometric Means,
*: Non-convergence caused by sparse data for AUC_{0-inf}

TABLE 12
Statistical Analysis of the Effect of Dosing Condition and High-fat meal for
Compound 1 Plasma Pharmacokinetic Parameters (Pharmacokinetic Analysis Set)

| | Fed Capsules | | | | | | | |
|---|---|---|---|---|---|---|---|---|
| | [high-fat meal] | | | Fasted Solution | | | Test/Reference | |
| Cₘₐₓ | 19 | 62.80 | (56.49, 70.87) | 21 | 114.30 | (99.19, 131.72) | 0.549 | (0.48, 0.63) |
| (ng/mL) | | | | | | | | |
| AUC₀₋ₜ | 19 | 834.06 | (766.99, 901.60) | 21 | 776.27 | (699.62, 861.31) | 1.074 | (1.02, 1.12) |
| (h*ng/mL) | | | | | | | | |
| AUC_{0-inf} | 4 | 866.90 | * | 5 | 770.31 | * | 1.125 | * |
| (h*ng/mL) | | | | | | | | |

In this analysis, a linear mixed model applied to the Compound 1 PK parameters AUC_{0-inf}, AUC₀₋ₜ, and Cₘₐₓ, will be used to test the effect of high-fat meal and dosing condition on the rate and extent of absolution. An unstructured covariance matrix will be used. The Compound 1 fasted solution condition will be considered the reference and the Compound 1 fed capsules [high-fat meal] will be the test treatment. Evidence of an absence of dosing condition and high-fat meal effect will be concluded if the 90% CI for AUC and Cₘₐₓ are contained within the interval (0.8, 1.25).
[1] Shows the number of subjects exposed to each treatment condition that were used in the mixed model.
GM = Geometric means, GMR = Ratio of Geometric Means,
*: Non-convergence caused by sparse data for AUC_{0-inf}

TABLE 13
Statistical Analysis of the Effect of Dosing Condition and Standard meal for
Compound 1 Plasma Pharmacokinetic Parameters (Pharmacokinetic Analysis Set)

| | Fed Capsules | | | | | | | |
|---|---|---|---|---|---|---|---|---|
| | [Standard meal] | | | Fasted Solution | | | Test/Reference | |
| Cₘₐₓ | 19 | 63.12 | (56.34, 72.62) | 21 | 114.30 | (99.19, 131.72) | 0.552 | (0.49, 0.64) |
| (ng/mL) | | | | | | | | |
| AUC₀₋ₜ | 19 | 837.55 | (763.47, 911.27) | 21 | 776.27 | (699.62, 861.31) | 1.079 | (1.02, 1.13) |
| (h*ng/mL) | | | | | | | | |
| AUC_{0-inf} | 5 | 1013.04 | * | 5 | 770.31 | * | 1.315 | * |
| (h*ng/mL) | | | | | | | | |

In this analysis, a linear mixed model applied to the Compound 1 PK parameters AUC_{0-inf}, AUC₀₋ₜ, and Cₘₐₓ, will be used to test the effect of standard-fat meal and dosing condition on the rate and extent of absorption. An unstructured covariance matrix will be used The Compound 1 fasted solution condition will be considered the reference and the Compound 1 fed capsules [standard-fat meal] will be the test treatment. Evidence of an absence of dosing condition and nigh-fat meal effect will be concluded if the 90% CI for AUC and Cₘₐₓ are contained within the interval (0.8, 1.25).
[1] Shows the number of subjects exposed to each treatment condition that were used in the mixed model.
GM = Geometric means, GMR = Ratio of Geometric Means,
*: Non-convergence caused by sparse data for AUC_{0-inf}

In the fasted state, the rate and extent of exposure of Compound 1 was greater for the solution than the capsules. The geometric mean ratios for Cₘₐₓ and AUC (capsules/solution) were 0.191 and 0.682, with the respective 90% CIs of (0.17, 0.22) and (0.62, 0.75).

The rate of exposure of Compound 1 was lower for the capsules in the fed state (high-fat meal) compared with the solution in the fasted state. The geometric mean ratio for Cₘₐₓ (90% CI) (fed/fasted) was 0.549 (0.48, 0.63). The extent of exposure for the capsule in the fed state (high-fat meal) was comparable to the extent of exposure of the solution in the fasted state. The geometric mean ratio for AUC₀₋ₜ (90% CI) was 1.074 (1.02, 1.12).

The rate of exposure of Compound 1 was lower for the capsules administered in the fed state (standard meal) compared with the solution in the fasted state. The geometric mean ratio for Cₘₐₓ (90% CI) (fed/fasted) was 0.552 (0.49, 0.64). The extent of exposure for the capsules in the fed state (high-fat meal) was comparable to the extent of exposure of the solution in the fasted state.

As shown in FIG. 1 and FIG. 2, Compound 1 capsules in the fed state (high-fat meal) have a nearly similar AUC curve as compared to the Compound 1 capsules in the fed state (standard meal). This is further supported by the data represented in Table 8, where the Mean AUC₀₋ₜ for Compound 1 capsules in the fed state (high-fat meal) is 846.5 h*ng/mL and the Mean AUC₀₋ₜ for Compound 1 capsules in the fed state (standard meal) is 851.8 h*ng/mL. Comparatively, the Mean AUC₀₋ₜ for Compound 1 capsules in the fasted state is 543.7 h*ng/mL.

The rate and extent of exposure of Compound 1 capsules was greater in the fed state (high-fat meal) compared with the fasted state. The geometric mean ratios for Cₘₐₓ and AUC (fed/fasted) were 2.879 and 1.575, with the respective 90% CIs of (2.56, 3.28) and (1.45, 1.69).

The rate and extent of exposure of Compound 1 capsules was greater in the fed state (standard meal) compared with the fasted state. The geometric mean ratios for Cₘₐₓ and AUC (fed/fasted) were 2.894 and 1.581, with the respective 90% CIs of (2.64, 3.25) and (1.43, 1.72).

### Example 2: Preparation of Solid Form A

Form A was prepared by stirring crude Compound 1 as a slurry in ethyl acetate below 10° C. and then filtering and drying under vacuum. It was also formed by dissolving crude Compound 1 in dichloromethane and then re-concentrating the solution twice with ethyl acetate under vacuum to dryness.

### Example 3: Various Wet Methods of Crystallization to Obtain Other Solid Forms of the Present Invention

To find new crystalline forms, different crystallization methods were evaluated using Form A as the starting material. In addition to Form A, Form C was identified with these methods.

### Slow Evaporation

Slow evaporation crystallization experiments were performed across 8 different solvent systems. In each experiment approximately 10 mg of Form A was dissolved in 0.4 to 1.0 mL of solvent in a 1.5 mL glass vial. The glass vials were sealed using Parafilm® pierced with 3 to 5 holes to allow for solvent evaporation.

### Slurry Conversion

In each experiment, approximately 10 to 20 mg of Form A was suspended in 0.5 mL of a solvent or mixture of solvents. After stirring at RT or 50° C. for 48 hours, the solids were isolated by centrifugation for analysis (wet sample). If the suspensions turned into clear solution, the clear solutions were kept at ambient conditions for slow evaporation.

### Anti-Solvent Addition

In each experiment, approximately 10 mg of Form A was dissolved in 0.1 to 1 mL of each solvent to obtain a clear solution. The anti-solvent was added in increments of 50 μL until precipitation was observed, or the total volume of anti-solvent reached 20 times that of the solvent volume. The precipitate was then isolated by centrifugation for analysis (wet sample). In the instances that clear solutions were observed, slow evaporation experiments were performed.

### Slow-Cooling

In each experiment, approximately 10 mg of Form A was suspended in 0.8 to 1.0 mL of each solvent mixture at 50° C. The resulting suspensions were immediately filtered with a 0.2 μm filter, and the filtrates were collected and cooled from 50° C. to 5° C. at a rate of 0.1° C./min. The precipitates were then isolated by centrifugation at 10,000 rpm for 3 to 5 minutes for analysis (wet sample).

### Solution Vapor Diffusion

In each experiment, approximately 10 mg of Form A was dissolved in an appropriate solvent to obtain a clear solution in a 3-mL glass vial. The vial was then placed into a 20-mL glass vial containing 3 mL of the anti-solvent and sealed. The system was kept at RT for 7 days, allowing sufficient time for solid precipitation. The solids were isolated by centrifugation at 10,000 rpm for 3 to 5 minutes and analyzed (wet sample). In the cases where no precipitation was observed, the samples were kept at ambient conditions for slow evaporation.

### Solid Vapor Diffusion

In each experiment, approximately 10 mg of Form A was placed into a 3-mL glass vial, which was then sealed into a 20-mL glass vial containing 3 mL of the specific solvent. The system was kept at RT for 7 days, allowing sufficient time for organic vapor to interact with the solids. The solids were then analyzed (wet sample).

### Fast Evaporation

In each experiment, approximately 10 mg of Form A was dissolved in 0.5 to 1.0 mL of each solvent in a 1.5-mL glass vial. The visually clear solutions were kept at ambient conditions for fast evaporation with the lid off. The solids obtained via evaporation were then analyzed (dry sample).

### Reverse Anti-Solvent Addition

In each experiment, approximately 20 mg of Form A was dissolved in 0.2 to 0.6 mL of each solvent to obtain a clear solution. The solution was added to a glass vial containing 2.0 mL of each anti-solvent at RT conditions. The precipitate formed was centrifuged at 10,000 rpm for 3 to 5 minutes for analysis (wet sample). In the cases where no precipitation was observed, slow evaporation experiments were conducted for the remaining solutions.

### Water Activity Experiments

Water activity experiments, ranging from 0 to 1 water activity (aw) at 0.2 intervals, were performed with H₂O and acetonitrile. About 10 mg of Form A was weighed into 1.5 mL vials and 0.5 mL of the solvent mixture was added. The suspension was stirred at a rate of 1000 rpm at room temperature. The residual solvent was removed from the sample by centrifugation (10000 rpm for 3 min).

### Example 4. Preparation of Solid Form C

Form C was prepared from Form A via a slurry conversion crystallization technique in isopropyl alcohol (IPA) and isopropyl acetate (IPAc) at 50° C.

### Example 5. Preparation of Solid Form K

Form K was prepared by heating Forms A, B, C, E, or F to elevated temperatures. The sample of Form K analyzed was prepared by heating Form F to 100° C.

### Example 6. Characterization of Solid Forms A and C by XRPD

A PANalytical Empyrean X-ray powder diffractometer with a 12-well auto-sampler stage was used for analysis throughout this study. The XRPD parameters used are listed in Table 14. Resolution calibration of the instrument was performed every 6 months, and sensitivity measurements were performed after the sample stage was changed. A silicon (Si) pressed powder sample was used as the reference standard.

TABLE 14
Parameters for XRPD

| | Parameters for Reflection Mode |
|---|---|
| X-Ray wavelength | Cu, kα, Kα1 (Å): 1.540598, |
| | Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (degrees 20) | 3° to 40° |
| Step size (degrees 20) | 0.017° |
| Scan speed (degrees/min) | ~10 |

Form A: Form A was observed to be crystalline by XRPD, as shown in FIG. 3A.

Form C: The XRPD pattern in FIG. 4A shows that Form C is crystalline.

### Example 7. Methods of Producing Single Crystals of Form A and Form C

Form A: Single crystals suitable for structure determination were obtained via slow cooling in isopropyl alcohol from 50° C. to 5° C.

Form C: Single crystals suitable for structure determination were obtained via slow cooling at a rate of 0.01° C./min in isopropyl acetate/acetone (6:1, v/v) co-solvents with Form C seeds, from 25° C. to 5° C.

### Example 8. Single Crystal X-Ray Diffraction Data of Form A and Form C

X-ray intensity data from prism-like crystals of Form A (Table 15) and Form C (Table 16) were collected at 290(2) K using a Bruker D8 Venture diffractometer (Mo Kα radiation, λ=0.71073 Å). The crystal structures of Forms A and C were solved from the obtained data.

TABLE 15
Crystal data and structural refinement
for a single crystal of Form A:

| Empirical formula | C₂₅H₃₅N₃O₂ |
|---|---|
| Formula weight | 409.56 |
| Temperature | 100(2)K |
| Wavelength | 0.71073 Å |
| Crystal system, space group | MonoclinicP2₁ |
| Unit cell dimensions | a = 9.379(3) Å |
| | b = 9.922(3) Å |
| | c = 12.092(4) Å |
| | α = 90° |
| | β = 101.606(9) ° |
| | γ = 90° |
| Volume | 1102.2(6) Å³ |
| Z, Calculated density | 2, 1.234 Mg/m³ |
| Absorption coefficient | 0.079 mm−1 |
| F(000) | 444 |
| Crystal size | 0.30 × 0.20 × 0.10 mm³ |
| Theta range for data collection | 2.22-27.56° |
| Limiting indices | −12 ≤ h ≤ 12 |
| | −12 ≤ k ≤ 12 |
| | −15 ≤ l ≤ 15 |
| Reflections collected/unique | 23466/5060 [R(int) = 0.0670] |
| Completeness | 99.9% |
| Refinement method | Full-matrix least-squares on F2 |
| Data/restraints/parameters | 5060/1/274 |
| Goodness-of-fit on F² | 1.071 |
| Final R indices [I > 2sigma(I)] | R₁ = 0.0425 |
| | wR₂ = 0.0989 |
| Largest diff. peak and hole | 0.309and −0.368 e.Å⁻³ |
| Absolute structure parameter | 1.5(11) |

TABLE 16
Crystal data and structural refinement
for a single crystal of Form C:

| Empirical formula | C₂₅H₃₅N₃O₂ |
|---|---|
| Formula weight | 409.56 |
| Temperature | 290(2) K |
| Wavelength | 0.71073 Å |
| Crystal system, space group | Orthorhombic P2₁2₁2₁ |
| Unit cell dimensions | a = 9.6642(8)Å |
| | b = 9.8676(8) Å |
| | c = 23.9408(19) Å |
| | α = 90° |
| | β = 90° |
| | γ = 90° |
| Volume | 2283.1(3)Å³ |
| Z, Calculated density | 4, 1.192 mg/m³ |
| Absorption coefficient | 0.076 mm⁻¹ |
| F(000) | 888 |
| Crystal size | 0.28 × 0.05 × 0.03 mm³ |
| Theta range for data collection | 2.71-27.61° |
| Limiting indices | −12 ≤ h ≤ 12 |
| | −12 ≤ k ≤ 12 |
| | −31 ≤ l ≤ 31 |
| Reflections collected/unique | 33905/5265 [R(int) = 0.0823] |
| Completeness | 99.3% |
| Refinement method | Full-matrix least-squares on F² |
| Data/restraints/parameters | 5265/7/272 |
| Goodness-of-fit on F² | 1.042 |
| Final R indices [I > 2sigma(I)] | R₁ = 0.0647 |
| | wR₂ = 0.1128 |
| Largest diff peak and hole | 0.248 and −0.335 e.A⁻³ |
| Absolute structure parameter | 0.0(19) |

### Example 9. Unit Cells of the Single-Crystal Structures of Form A and Form C

The unit cell of Form A along the b axis is depicted in FIG. 3B. The unit cell of Form C along the b axis is depicted in FIG. 4B.

### Example 10. Characterization of Solid Forms a, C, and K by Temperature-Dependent Instrumental Methods (TGA, DSC, and VT-XRPD)

Thermogravimetric analysis (TGA) data were collected using a TA Q500/Q5000 TGA from TA Instruments, and differential scanning calorimetry (DSC) was performed using a TA Q200/Q2000 DSC from TA Instruments. The instrument parameters used are provided in Table 17.

TABLE 17
Parameters for TGA and DSC Test

| Parameters | TGA | DSC |
|---|---|---|
| Method | Ramp | Ramp |
| Sample pan | Platinum, open | Aluminum, crimped |
| Temperature | RT to 350° C. | RT to 300° C. |
| Heating rate | 10° C./min | 10° C./min |
| Purge gas | N₂ | N₂ |

To complement the temperature-dependent studies and confirm the solvation state of the solid forms, solution NMR was collected on a Bruker 400 MHz NMR Spectrometer using deuterated dimethyl sulfoxide (DMSO-d6) as the solvent.

Form A: TGA and DSC were performed and the details provided in FIG. 3C. Thermogravimetric analysis of Form A resulted in a 1.0% weight loss up to 200° C. An endotherm observed on the DSC curve at 157.2° C. (onset temperature), representing the transformation of Form A to Form K, was followed by a sharp melting peak for Form K at 203.8° C. (onset temperature). Verification of the transformation to Form K was performed by VT-XRPD, as shown in FIG. 3D.

Form C: TGA and DSC were performed, and their respective curves are provided in FIG. 4C. The TGA curve shows that a weight loss of 4.3% occurs below 50° C. indicating loosely held solvent or adventitious solvent, possibly present due to insufficient drying. The DSC curve exhibits 2 endothermic peaks at 183.8° C. and 211.0° C. (onset temperatures). Further investigation of the endotherm at 183.8° C. was performed by heating Form C to 185° C., which resulted in a form transformation to Form K, as shown in FIG. 4D. Analysis by VT-XRPD was performed on Form C, with and without nitrogen (N₂) flow, to investigate possible rehydration from air. As shown in FIG. 4E, no differences were observed with and without N₂, indicating that Form C is an anhydrate.

### Example 11. Hygroscopicity of Forms A, C, and K as Measured by DVS

Dynamic vapor sorption (DVS) was measured via an SMS (Surface Measurement Systems) DVS Intrinsic system. The relative humidity at 25° C. was calibrated against the deliquescence point of LiCl, Mg(NO₃)₂, and KCl. Instrument parameters for the DVS system used throughout this study are listed in Table 18.

TABLE 18
Parameters for DVS Test

| Parameters | DVS | |
|---|---|---|
| Temperature | 25° | C. |
| Sample size | 10-20 | mg |
| Gas and flow rate | N₂, 200 mL/min | |
| dm/dt | 0.002%/min | |
| Min. dm/dtstabilityduration | 10 | min |
| Max. equilibrium time | 180 | min |

| | |
|---|---|
| RH range | 0% RH to 95% RH |
| RH step size (sorption) | 10% RH from 0% RHto 90% RH |
| | 5% RH from 90% RH to 95% RH |
| RH step size (desorption) | 10% RH from 90% RH to 0% RH |
| | 5% RH from 95% RH to 90% RH |

The hygroscopicity of Form A, Form C, and Form K were investigated at 25° C. using DVS. The XRPD patterns of each sample before and after DVS were compared in order to investigate any potential form change.

The DVS isotherm plot of Form A shown in FIG. 3E exhibits 0.06% by weight water uptake at 80% RH and less than 0.12% by weight water uptake at 95% RH, revealing that Form A is non-hygroscopic. The XRPD pattern in FIG. 3F indicates there is no form change before and after DVS for Form A.

Similarly, the DVS isotherm plot of Form C shown in FIG. 4F exhibits 0.12% by weight water uptake at 80% RH and less than 0.30% by weight water uptake at 95% RH, indicating that Form C is non-hygroscopic. The XRPD pattern in FIG. 4G shows there is no form change before and after DVS for Form C.

### Example 12. Interconversion of Forms A, C, and K Through Slurry Conversion

In one embodiment, the inter-conversion between Forms A, C and K can be studied in a series of slurry conversion experiments conducted in ethyl acetate, n-butanol, and methyl tert-butyl ether (MBTE) at both room temperature (RT) and 50° C. Compound 1 can display moderate solubility, and may yield solvated forms during these screening experiments. Results of the slurry conversion experiments are summarized in Table 19. The transition temperature between Forms A and C was estimated to be ˜17° C., and the transition temperature between Forms K and C was above 100° C.

TABLE 19
Summary of Slurry Conversion Experiments

| Solvent | Condition | Initial Form | Final Form |
|---|---|---|---|
| Ethyl | RT | Forms A and K | Form C |
| acetate | | (with Form C seeds) | |
| | 50° C. | Forms A and K | Form C |
| n-Butanol | RT | Forms A, C and K | Form C |
| | 50° C. | Forms A, C and K | Form C |
| MBTE | RT | Forms A, C and K | Form C |
| | 50° C. | Forms A, C and K | Form C |

### Example 13. Conversion of Form A to Form C with Form C Seed Crystals

Approximately 200 g/L-225 g/L solubilized Compound 1 (originally Form A) in ethyl acetate was heated to a temperature of 65° C. in the presence of 0.2%-1.0% of seed crystals of Form C for 1-3 hours. The batch can then be slowly cooled down to a temperature between 25° C.−30° C. for no less than 3 hours to obtain Form C. Seed crystals of Form C can be obtained using the procedure described in Example 4.

XRPD was performed using a Rigaku MiniFlex 600 (Cu Kα radiation at 40 kV tube voltage and 15 mA tube current) with a scanning range of 2° to 40° for 2θ, a step size of 0.01°, and a scanning speed of 1° or 2° per minute. XRPD was used to monitor the conversion from 225 g/L Form A to Form C in ethyl acetate at 65° C. using 1.0% of seed crystals of Form C with time, as indicated in FIG. 6.

### Example 14. Rapid and Sustained Improvement in Depressive Symptoms in a Phase 2, Open-Label Study Evaluating Compound 1 in Subjects with Major Depressive Disorder (“Part A”)

In this multicenter, open-label, Phase 2 clinical trial, subjects were administered 30 mg Compound 1 on Days 1-14 and assessed through Day 28. Reductions in depressive and anxiety symptoms were assessed by the Hamilton Rating Scales for Depression and Anxiety (HAM-D and HAM-A), and the Montgomery-Asberg Depression Rating Scale (MADRS). Safety and tolerability were assessed via standard safety parameters. Pharmacokinetic parameters were also evaluated.

### Methods and Materials:

### Study Design and Participants:

This multicenter, open-label component of the Phase 2 trial was conducted at 2 sites in the United States, with IRB approval at each site. Sage Therapeutics, Inc. collaborated with the principal investigator (RR) in the design of the trial and all investigators in the execution of the trial and collection of data. All authors vouch for the accuracy and completeness of the data, data analyses, and the fidelity of this publication to the study protocol.

### Study Population:

Written informed consent was provided at screening and was required for enrollment. The diagnosis of MDD was made using the Structured Clinical Interview for Diagnostic and Statistical Manual of Mental Disorders, Fifth Edition (DSM-5) Axis I Disorders (SCID-I), as performed by a qualified healthcare professional. Subjects were also screened with the 17-item Hamilton Rating Scale for Depression (HAM-D) to ensure symptom severity of at least 22. Hamilton Anxiety Rating Scale (HAM-A), Montgomery-Asberg Depression Rating Scale (MADRS), and Clinical Global Impression-Severity (CGI-S) scale were also administered. Using the 17-item HAM-D scale, a patient with a summed score of 17-23 was considered to have moderate depression; a summed score of ≥24 was considered severe depression. Patient histories and concomitant medication information were collected. The study population included subjects of both sexes, ages 18-65 years old, inclusive (inclusion/exclusion criteria below). Subjects remained as inpatients during the first 7 days of the study period and per Investigator's judgement thereafter. Subjects received the standard of care for adult inpatients diagnosed with MDD and were allowed to remain on a stable dose of psychotropic medications that were initiated at least 14 days prior to screening.

Exclusion criteria included: history of suicide attempt, history of treatment-resistant depression, recent history or active clinically significant manifestations of other acute or chronic conditions, positive pregnancy test, history of seizures, medical history of bipolar disorder, schizophrenia, and/or schizoaffective disorder. A full list of exclusion criteria are provided in the Supplementary Appendix.

### Procedures:

Compound 1 was provided as a 6 mg/mL stock aqueous solution, which was further diluted with sterile water to achieve the selected dose that was administered as an oral solution. Participants received an open label 30 mg Compound 1 oral dose administered at 8:00 PM (+/−15 minutes) with food on Days 1 through 14. Subjects who experienced drug-related moderate or severe adverse events (as judged by the study investigators) while receiving the 30 mg dose of study drug were to have their dose reduced to 20 mg for the remaining treatment period. Any subjects not tolerating a 20 mg dose were to be terminated from the study. Vital signs were collected daily. Other study procedures and plasma collection for PK parameters were conducted at various times during the 14-day open label treatment period (procedure details in the Supplemental Appendix). Treatment follow-up was conducted on an outpatient basis and included a follow-up visit at 1 week (21+/−1 day) and at 2 weeks (28+/−1 day) after the last dose of the study drug.

### Outcomes:

The primary endpoint of safety and tolerability of Compound 1 was assessed by the frequency and severity of adverse events, vital signs, changes in clinical laboratory measures, physical exams, electrocardiograms (ECGs), the Stanford Sleepiness Scale (SSS) score, and suicide ideation using the Columbia-Suicide Severity Rating Scale (C-SSRS, Supplemental Table 1). Secondary efficacy endpoints included: change from baseline in HAM-D total score, HAM-D response, defined as having a 50% or greater reduction from baseline in HAM-D total score, HAM-D remission, defined as having a HAM-D total score of ≤7, HAM-A change from baseline, and MADRS score change from baseline. The PK profile of Compound 1 was also determined from plasma samples.

### Statistical Analysis:

Continuous parameters were summarized as n, mean, and standard deviation, with categorical variables summarized as frequency counts and percentages. For HAM-D, HAM-A, and MADRS total scores, p-values for change from baseline values were calculated based on mixed effects model for repeated measures with visit as fixed effect, adjusting for baseline total score. All statistical analyses were performed using SAS® statistical software, version 9.3 (SAS Institute Inc, Cary, N.C.).

### Results:

### Subjects

This study enrolled 13 subjects with a mean (SD) age of 48.0 (12.8), and range of 20-64 years old. Subjects' mean (SD) baseline values for HAM-D, HAM-A, and MADRS were 27.2 (3.1), 23.2 (5.7), and 36.9 (5.2), respectively (Table 20). All 13 subjects who enrolled in the study completed the study. The demographic characteristics of the subjects are summarized in Table 20.

TABLE 20
Subject Characteristics and Demographics

| | Total | |
|---|---|---|
| Subjects Characteristics | (N = 13) | |
| Age (years) | | |
| Mean (SD) | 48.0 | (12.8) |
| Gender | | |
| Male | 4 | (30.8%) |
| Female | 9 | (69.2%) |
| Race | | |
| White | 4 | (30.8%) |
| Black or African American | 9 | (69.2%) |
| Ethnicity | | |
| Hispanic or Latino | 1 | (7.7) |
| Not Hispanic or Latino | 12 | (92.3%) |
| Baseline HAM-D | | |
| Mean (SD) | 27.2 | (3.1) |
| Baseline HAM-A | | |
| Mean (SD) | 23.2 | (5.7) |
| Baseline MADRS | | |
| Mean (SD) | 36.9 | (5.2) |
| Subjects with at least one prior medication | 10 | (76.9%) |
| Subjects with at least one concomitant medication | 11 | (84.6) |

### Efficacy

Compound 1 administration produced rapid and sustained reductions across all depression endpoints. Compound 1 significantly reduced depressive symptoms assessed by the HAM-D total score, decreasing from a mean total score of 27.2 at baseline to 7.3 at Day 15, a mean decrease of 19.9 points (p<0.0001) (FIG. 1A). Significant reductions in depressive symptoms were seen as early as Day 2 (p<0.05). (FIG. 1A). These changes were maintained through the conclusion of the study, which was two weeks after study drug discontinuation (Day 28, p<0.0001) (FIG. 1A). The reduction in depressive symptoms assessed by the MADRS total score was consistent with the pattern observed for the HAM-D, with a decrease from a mean total score of 36.9 at baseline to 10.5 at Day 15, a mean decrease of 26.4 points (p<0.0001) (FIG. 1B). The decrease in MADRS was sustained through Day 28 (p<0.0001) (FIG. 1B). Compound 1 also reduced symptoms of anxiety assessed by the HAM-A total score, with a decrease from a mean total score of 23.2 at baseline to 7.7 at Day 15, a mean decrease of 15.5 points (p<0.0001) (FIG. 1C). These changes were sustained to the conclusion of the study (Day 28, p<0.0001) (FIG. 1C).

In 11 of 13 subjects (85%), the HAM-D total score was reduced by least 50% (defined as HAM-D response) at Day 15 (FIG. 2), and this HAM-D response was sustained through Day 28. The treatment effect was durable, as changes were maintained throughout the treatment period and maintained after treatment discontinuation. The HAM-D response rates on Days 15 and 28 were high regardless of the presence (5/5, 100%, Day 15; 4/5, 80%, Day 28) or absence (6/8; 75.0%, Day 15; 6/8, 75%, Day 28) of antidepressants at the time of enrollment.

In 8 of 13 subjects (62%), HAM-D total score was <7 at the end of the treatment period (defined as HAM-D remission) and this HAM-D remission was sustained through Day 28 (FIG. 2). Remission was 39% by Day 6 and the remission rate remained stable after treatment was discontinued between Day 15 and Day 28 (FIG. 2).

### Safety and Pharmacokinetics

Compound 1 was generally well tolerated and there were no serious adverse events (SAEs) or discontinuations due to AEs. Nearly all subjects experienced at least one treatment emergent adverse event (TEAE) (12/13; 92.3%; Table 2). The most common AEs were sedation (6 subjects), headache (4 subjects), somnolence (3 subjects), dizziness (3 subjects), and myalgia (3 subjects) (Table 21). Suicidal ideation was assessed by the Columbia-Suicide Severity Rating Scale (C-SSRS). On Days 10 through 13, one subject had engaged in non-suicidal self-injurious behavior since the last visit (Supplementary FIG. 1). There were no clinically relevant changes in clinical hematology, urinalysis, vital signs, or ECGs. There were no clinically relevant changes in clinical chemistry, with the exception of one subject that showed mild and transient elevations in ALT, AST, and alkaline phosphatase that resolved, were not associated with any clinical findings, and did not result in study drug dose adjustment or discontinuation.

TABLE 21
Treatment emergent adverse events (TEAEs).

Compound 1
(N = 13)

Overall Summary

| | | |
|---|---|---|
| At least one TEAE | 12 | (92.3%) |
| Drug-related TEAE | 11 | (84.6%) |

| | |
|---|---|
| Severe TEAE | 0 |
| Serious AE | 0 |
| TEAE leading to drug discontinuation | 0 |
| TEAE leading to death | 0 |

AEs in at least two subjects

| | | |
|---|---|---|
| Sedation | 6 | (46.2%) |
| Headache | 4 | (30.8%) |
| Dizziness | 3 | (23.1%) |
| Somnolence | 3 | (23.1%) |
| Myalgia | 3 | (23.1%) |
| Nasal Congestion | 2 | (15.4%) |

Pharmacokinetic (PK) parameters were derived from plasma samples (Table 22). PK and exposure data were consistent with those previously observed in Phase 1 Compound 1 studies in healthy volunteers (SAD/MAD reference). Mean Compound 1 plasma concentrations reached a maximum at a median Tₘₐₓ of 1.1 hours after administration of Compound 1 with a half-life of 10.3 hours. At steady state, the geometric mean Cₘₐₓ and C_{avg,ss} were 101.0 ng/mL and 41.4 ng/mL, respectively. Similarly, at steady state, the geometric mean values for AUC₀₋ₜ and AUC_{inf} were 992.5 h·ng/mL and 1150.7 h·ng/mL, respectively.

TABLE 22
Pharmacokinetic Parameters, Day 7. CV % =
coefficient of variation. N = 13

| Parameter | | |
|---|---|---|
| | Geometric Mean (Geometric CV %) | |
| Cₘₐₓ (ng/mL) | 101.0 | (37.9) |
| AUC_{inf} (hr*ng/mL) | 1150.7 | (21.6) |
| C_{avg} ₛₛ (ng/mL) | 41.4 | (24.3) |
| AUC₀₋ₜ (hr*ng/mL) | 992.5 | (24.3) |

| | Median |
|---|---|
| T_{1/2} (hr) | 10.3 |
| Tₘₐₓ (hr) | 1.1 |

### Inclusion Criteria:

1. Subject has signed an ICF prior to any study-specific procedures being performed.
2. Subject is an ambulatory male or female between 18 and 65 years of age, inclusive.
3. Subject is in good physical health and has no clinically significant findings, as determined by the Investigator, on physical examination, 12-lead ECG, or clinical laboratory tests.
4. Subject agrees to adhere to the study requirements.
5. Subject has a diagnosis of MDD that has been present for at least a 4-week period as diagnosed by SCID-I.
6. Subject has a HAM-D total score of ≥22 at screening and Day 1 (prior to dosing).
7. Subject is willing to delay the start of other antidepressant or antianxiety medications and any new pharmacotherapy regimens, including as-needed benzodiazepine anxiolytics, during the screening and treatment periods.
8. Subject agrees to practice an acceptable method of highly effective birth control at screening and throughout study participation. Highly effective methods of birth control include sexual abstinence (for males and females); vasectomy; or a condom with spermicide in combination with a highly effective female partner's method (for males); and hormonal methods of contraception (i.e., established use of oral, implantable, injectable, or transdermal hormones); placement of an intrauterine device; placement of an intrauterine system; and mechanical/barrier method of contraception (i.e., condom or occlusive cap [diaphragm or cervical/vault cap] in conjunction with spermicide [foam, gel, film, cream, or suppository]) (for females).

### Exclusion Criteria:

1. Subject has a history of suicide attempt.
2. Subject has a recent history or active clinically significant manifestations of metabolic, hepatic, renal, hematological, pulmonary, cardiovascular, gastrointestinal, musculoskeletal, dermatological, urogenital, neurological, or eyes, ears, nose, and throat disorders, or any other acute or chronic condition that, in the Investigator's opinion, would limit the subject's ability to complete or participate in this clinical study.
3. Subject has a history of treatment-resistant depression, defined as persistent depressive symptoms despite treatment with adequate doses of antidepressants from two different classes for an adequate amount of time (i.e., at least 4 weeks of treatment).
4. Subject has a known allergy to Compound 1, allopregnanolone, or related compounds.
5. Subject has a positive pregnancy test at screening or on Day 1 prior to the start of study drug administration.
6. Subject has detectable hepatitis B surface antigen (HBsAg), anti-hepatitis C virus (HCV), or human immunodeficiency virus (HIV) antibody at screening.
7. Subject has active psychosis per Investigator assessment.
8. Subject has a medical history of seizures.
9. Subject has a medical history of bipolar disorder, schizophrenia, and/or schizoaffective disorder.
10. Subject has a history of alcohol or drug dependence (including benzodiazepines) in the 12 months prior to screening.
11. Subject has had exposure to another investigational medication or device within 30 days prior to screening.
12. Subject has been treated or randomized in this study (e.g., Part A) or any other study employing Compound 1 previously (i.e., subject may not have received study drug and then re-enroll).
13. Subject has had administration of psychotropics that have been initiated within 14 days prior to screening and/or are not being taken at a stable dose.
14. Use of any known strong inhibitors and/or inducers of cytochrome P450 (CYP)3A4 within the 14 days or five half-lives (whichever is longer) or consumed grapefruit juice, grapefruit, Seville oranges, or St. John's Wort or products containing these within 30 days prior to receiving the first dose of study drug.
15. Subject has a positive drug and/or alcohol screen at screening or on Day 1 prior to dosing.

### Procedure Details:

Part A of the study consisted of an up to 7-day Screening Period (Days −7 to −1), a 14-day Treatment Period, and a 2-week Follow-up Period (through Day 28). During the Screening Period (Day −7 to Day −1), after signing the informed consent form (ICF), subjects were be assessed for study eligibility, and the severity of each subject's MDD was evaluated using HAM-D. The Screening Period assessments were conducted on an outpatient basis.

If applicable, standard of care data collected prior to obtaining informed consent included as screening data, if appropriate, such as laboratory tests, ECG, physical examination, and vital signs conducted within the preceding 48 hours, as long as the requirement for the screening assessment to be collected retrospectively is met in full. If applicable, to ensure protocol compliance, any standard of care data eligible for inclusion as screening data must include the precise nature and timing of data collection.

During the 14-day study Treatment Period subjects remained inpatient for the first 7 days at minimum and per Investigator's judgement thereafter. The Follow-up Period assessments was conducted on an outpatient basis.

The study was conducted as follows:

Beginning on Day 1, subjects received open-label Compound 1 Oral Solution at 8:00 PM (±15 minutes) with food (as outlined in Section 9.2.1). Subjects received Compound 1 Oral Solution 30 mg from Day 1 to Day 14 as tolerated. Study drug (Compound 1 Oral Solution or matching placebo) was administered at the study center for at least the first 7 days of the Treatment Period, which includes Day 1 of study drug administration through completion of study drug administration on Day 14.

Subjects were discharged after a minimum 7-day inpatient stay, following completion of the Day 7 assessments. If their clinical condition did not allow discharge, the Investigator kept the subjects as inpatients for a longer period of time. Subjects discharged from the inpatient unit received treatment with study drug for the remainder of the 14-day Treatment Period as outpatients. For the outpatient phase, dosing will be done at the clinical site or, if suitable arrangements can be made, via home administration where local regulations allow. Home administration of study drug was performed according to a site-specific plan by a healthcare professional trained on the protocol and delivery of the study drug.

Subjects were monitored for safety during the Treatment and Follow-up Periods including monitoring for adverse events/serious adverse events, routine clinical laboratory assessments, physical examination, vital signs, and ECG. During the Treatment Period, subjects received study drug as long as there are no dose-limiting safety/tolerability concerns

SUPPLEMENTAL TABLE 1
Columbia Suicide Severity Rating Scale: Suicidality was monitored
during the study using the C-SSRS. This scale consists of an
evaluation that assesses the lifetime and recent experience of
the subject with suicidal ideation and behavior. N = 13.

| | Lifetime | Past 24 months | Day 1 | Day 2-15 |
|---|---|---|---|---|
| Suicide | Number of | Number of | Number of | Number of |
| Behavior | subjects who | subjects who | subjects who | subjects who |
| Item | answered yes | answered yes | answered yes | answered yes |
| Actual | 0 | 0 | 0 | 0 |
| attempt | | | | |
| Subject | 0 | 0 | 0 | 1* |
| engaged in | | | | |
| non-suicidal | | | | |
| self- | | | | |
| injurious | | | | |
| behavior | | | | |
| Interrupted | 0 | 0 | 0 | 0 |
| attempt | | | | |
| Aborted | 0 | 0 | 0 | 0 |
| attempt | | | | |
| Preparatory | 0 | 0 | 0 | 0 |
| acts or | | | | |
| behavior | | | | |
| Suicidal | 0 | 0 | 0 | 0 |
| behavior | | | | |

*One subject answered “yes” on Days 10-13.

### Example 15. A Phase 2, Double-Blind, Placebo-Controlled Clinical Trial of Compound 1 in the Treatment of Adult Patients with Moderate to Severe Major Depressive Disorder (MDD) (“Part B”)

In the randomized, double-blind, parallel-group, placebo-controlled trial, eligible patients (with a minimum total score of 22 on the Hamilton Rating Scale for Depression) were stratified based on use of antidepressant treatment (current/stable or not treated/withdrawn ≥30 days) and randomized in a 1:1 ratio to receive Compound 1 Capsules (30 mg) or matching placebo. All doses of study drug were administered at night with food. The study consisted of a 14-day treatment period, and a 4-week follow-up period. The mean HAM-D total scores at baseline were 25.2 for the Compound 1 group and 25.7 for the placebo group (overall range 22-33), representing patients with moderate to severe MDD. Approximately 90 percent of patients in each group completed the study.

In this trial, treatment for 14 days with Compound 1 was associated with a statistically significant mean reduction in the Hamilton Rating Scale for Depression (HAM-D) total score from baseline to Day 15 (the time of the primary endpoint) of 17.6 points compared with a 10.7 point mean reduction in HAM-D total score associated with placebo (least squared mean difference from placebo of −7.0; p<0.0001). Improvements in the HAM-D total score compared to placebo were significant by the morning following the first dose (Day 2) and were durable through the end of follow-up at week 6, with statistical significance noted through week 4. At Day 15, 64 percent of patients who received Compound 1 achieved remission, defined as not more than 7 on the HAM-D total score compared with 23 percent of patients who received placebo (p=0.0005). Other secondary endpoints were all similarly highly significant at Day 15 (p<0.0021).

Compound 1 was generally well-tolerated with no serious or severe adverse events; the most common AEs were headache, dizziness, nausea, and somnolence. A low rate of discontinuations due to adverse events (AEs) was reported; overall reports of AEs were similar between drug (53%) and placebo (46%), with a safety profile consistent with that seen in earlier trials.

Summary of Top-Line Results from the Placebo-Controlled Phase 2 Trial

### Effect on Depressive Symptoms Through End of Treatment (Day 15):

Treatment with Compound 1 was associated with a statistically significant mean reduction from baseline in the Hamilton Rating Scale for Depression (HAM-D) total score at Day 15 of 17.6 points compared with a 10.7 point mean reduction in HAM-D total score associated with placebo (p<0.0001).
The majority of patients (64%) who received Compound 1 achieved remission at Day 15 as determined by a HAM-D total score less than or equal to 7 (compared with 23% of patients who received placebo, p=0.0005).
Other secondary endpoints (e.g., MADRS, CGI-I) were similarly highly significant at Day 15 (p<0.0021).

### Effect on Depressive Symptoms Over Time:

Statistically significant mean reductions from baseline in the Hamilton Rating Scale for Depression (HAM-D) total score was observed following the first dose (Day 2) and maintained through Week 4, two weeks after end of treatment (p<0.0318).
At Week 4, the mean reduction from baseline in HAM-D total score was 15.6 for the Compound 1 group and 11.9 for the placebo group (p=0.0243).
At Week 6, the mean reduction in HAM-D total score for the Compound 1 group was 15.0 and numerically, but not statistically improved compared to the placebo group reduction of 13.0.
Rates of remission at Week 4 and Week 6 for patients treated with Compound 1 were 52 percent and 45 percent compared to 28 percent and 33 percent for placebo, with statistical significance maintained at Week 4 (p=0.0221) but not Week 6.

### Safety and Tolerability:

Compound 1 was generally well tolerated in the trial. Overall incidence of patients who experienced adverse events was 53 percent for the Compound 1 treatment group and 46 percent for the placebo group.
There were no deaths, serious or severe adverse events.
Rates of discontinuation from dosing of study drug due to adverse events were low; two patients (4.4%) treated with Compound 1 and none treated with placebo.

### Inclusion Criteria:

Subject has a diagnosis of Major Depressive Disorder that has been present for at least a 4-week period as diagnosed by Structured Clinical Interview for DSM-IV Axis I Disorders (SCID-I).

### Exclusion Criteria:

Subject has a history of suicide attempt
Subject has a history of treatment-resistant depression, defined as persistent depressive symptoms despite treatment with adequate doses of antidepressants from two different classes for an adequate amount of time
Active psychosis
Medical history of seizures
Medical history of bipolar disorder, schizophrenia, and/or schizoaffective disorder

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a CNS-related disorder in a subject in need thereof, the method comprising administering to the subject a therapeutically effective amount a compound having the formula

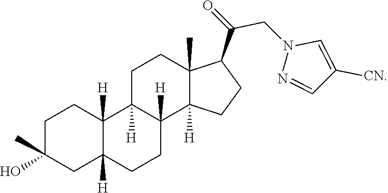

2. The method of claim 1, wherein the compound is administered without food or substantially contemporaneously with food.

3. The method of claim 1, wherein the compound is administered substantially contemporaneously with food.

4. The method of any one of claims 1-3, wherein the therapeutically effective amount is in a solid dosage form.

5. The method of any one of claims 1-4, wherein the solid dosage form is a tablet or a capsule.

6. The method of any one of claims 2-5, wherein administering the therapeutically effective amount of the compound substantially contemporaneously with food increases the bioavailability of the compound compared to administration without food, wherein bioavailability is based on a comparison of AUC values.

7. The method of claim 6, wherein the bioavailability is increased by about 10% or greater.

8. The method of claim 6, wherein the bioavailability is increased by about 20% or greater.

9. The method of claim 6, wherein the bioavailability is increased by about 30% or greater.

10. The method of claim 6, wherein the bioavailability is increased by about 40% or greater.

11. The method of claim 6, wherein the bioavailability is increased by about 50% or greater.

12. The method of claim 6, wherein the bioavailability is increased by about 55% or greater.

13. The method of claim any one of claims 1-12, wherein the therapeutically effective amount is administered once daily.

14. The method of any one of claims 1-13, wherein the therapeutically effective amount is about 20 mg to about 60 mg of the compound.

15. The method of any one of claims 1-13, wherein the therapeutically effective amount is about 25 mg to about 50 mg of the compound.

16. The method of any one of claims 1-13, wherein the therapeutically effective amount is about 25 mg to about 35 mg of the compound.

17. The method of any one of claims 1-13, wherein the therapeutically effective amount is about 30 mg, e.g., 30 mg.

18. The method of any one of claims 2-17, wherein the subject is administered the therapeutically effective amount of the compound within 60 minutes before or after ingesting the food.

19. The method of any one of claims 2-17, wherein the subject is administered the therapeutically effective amount of the compound within 45 minutes before or after ingesting the food.

20. The method of any one of claims 2-17, wherein the subject is administered the therapeutically effective amount of the compound within 30 minutes before or after ingesting the food.

21. The method of any one of claims 2-17, wherein the subject is administered the therapeutically effective amount of the compound within 15 minutes before or after ingesting the food.

22. The method of any one of claims 2-17, wherein the subject is administered the therapeutically effective amount of the compound within 5 minutes before or after ingesting the food.

23. The method of any one of claims 2-22, wherein the food comprises a high fat meal.

24. The method of any one of claims 2-22, wherein the food comprises a regular meal.

25. The method of any one of claims 2-22, wherein the food comprises at least 50 calories.

26. The method of any one of claims 2-22, wherein the food comprises at least 100 calories.

27. The method of any one of claims 2-22, wherein the food comprises at least 200 calories.

28. The method of any one of claims 2-22, wherein the food comprises at least 300 calories.

29. The method of any one of claims 1-28, wherein the CNS-related disorder is a GABA related disease or disorder.

30. The method of any one of claims 1-28, wherein the CNS-related disorder is a mood disorder.

31. The method of any one of claims 1-28, wherein the CNS-related disorder is a movement disorder.

32. The method of any one of claims 1-28, wherein the CNS-related disorder is postpartum depression.

33. The method of any one of claims 1-28, wherein the CNS-related disorder is major depressive disorder.

34. The method of any one of claims 1-28, wherein the CNS-related disorder is essential tremor.

35. The method of any one of claims 1-28, wherein the movement disorder is Parkinson's disease.

36. The method of claim 33, wherein the baseline HAM-D total score of the subject is at least 24.

37. The method of claim 33, wherein the baseline HAM-D total score of the subject is at least 18.

38. The method of claim 33, wherein the baseline HAM-D total score of the subject is at between and including 19 and 22.

39. The method of claim 33, wherein the baseline HAM-D total score of the subject is at between and including 14 and 18.

40. The method of claim 33, wherein the baseline HAM-D total score of the subject is greater than or equal to 23.

41. The method of any one of claims 36-40, wherein the compound is administered to the subject once a day for at least 14 days.

42. The method of any one of claims 36-41, wherein the HAM-D total score associated with treating the subject with the compound is no more than a number ranging from 6 to 8.

43. The method of any one of claims 36-42, wherein the HAM-D total score associated with treating the subject with the compound is no more than 7.

44. The method of any one of claims 36-42, wherein the decrease in HAM-D total score after treating the subject with Compound 1 relative to the baseline HAM-D total score is at least 15.

45. The method of any one of claims 36-42, wherein the decrease in HAM-D total score after treating the subject with Compound 1 relative to the baseline HAM-D total score is at least 17.

46. The method of any one of claims 1-45, wherein the subject is administered the compound at night.

47. The method of any one of claims 1-46, wherein the subject is administered the compound no longer than 1 hour before the subject sleeps.

48. The method of any one of claims 1-47, wherein the subject is administered the compound no longer than 15 minutes before the subject sleeps.

49. The method of any one of claims 1-48, wherein the compound is administered chronically.

50. An article of manufacture comprising:
a therapeutically effective amount of a compound having the formula 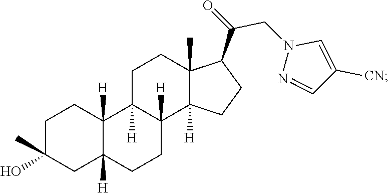
packaging material; and
a label affixed to the packaging material indicating that the therapeutically effective amount of the compound should be taken with food or a package insert contained within the packaging material indicating that the therapeutically effective amount of the compound should be taken with food.

51. The article of manufacture of claim 50, wherein the therapeutically effective amount of the compound is in a solid dosage form.

52. The article of manufacture of claim 51, wherein the solid dosage form is a tablet or a capsule.

53. The article of manufacture of claim 50, wherein the label or package insert further indicates that the therapeutically effective amount of the compound is administered once daily.

54. The article of manufacture of any one of claims 50-53, wherein the therapeutically effective amount of the compound is about 20 mg to about 60 mg.

55. The article of manufacture of any one of claims 50-53, wherein the therapeutically effective amount of the compound is about 25 mg to about 50 mg.

56. The article of manufacture of any one of claims 50-53, wherein the therapeutically effective amount of the compound is about 25 mg to about 35 mg.

57. The article of manufacture of any one of claims 50-53, wherein the therapeutically effective amount of the compound is about 30 mg, e.g., 30 mg.

58. The article of manufacture of any one of claims 50-57, wherein the food comprises a high fat meal.

59. The article of manufacture of any one of claims 50-57, wherein the food comprises a regular meal.

60. The article of manufacture of any one of claims 50-59, wherein the label further indicates that the therapeutically effective amount of the compound should be taken with food to increase bioavailability.

61. The article of manufacture of any one of claims 50-60, wherein the label or the package insert further indicates that the therapeutically effective amount of the compound is for treating CNS related disorders.

62. A method of effecting positive allosteric modulation of a GABA_{A} receptor in a patient in need thereof, comprising administering to the patient a therapeutically effective amount of a compound substantially contemporaneously with food, the compound having the formula

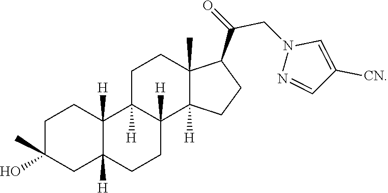

63. The method of claim 62, wherein the therapeutically effective amount is in a solid dosage form.

64. The method of claim 63, wherein the solid dosage form is a tablet or a capsule.

65. The method of claim 62, wherein the therapeutically effective amount is administered once daily.

66. The method of any one of claims 62-65, wherein the therapeutically effective amount is about 20 mg to about 60 mg of the compound.

67. The method of any one of claims 62-65, wherein the therapeutically effective amount is about 25 mg to about 50 mg of the compound.

68. The method of any one of claims 62-65, wherein the therapeutically effective amount is about 25 mg to about 35 mg of the compound.

69. The method of any one of claims 62-65, wherein the therapeutically effective amount is about 30 mg, e.g., 30 mg.

70. The method of any one of claims 62-69, wherein the subject is administered the therapeutically effective amount of the compound within 60 minutes before or after ingesting the food.

71. The method of any one of claims 62-69, wherein the subject is administered the therapeutically effective amount of the compound within 45 minutes before or after ingesting the food.

72. The method of any one of claims 62-69, wherein the subject is administered the therapeutically effective amount of the compound within 30 minutes before or after ingesting the food.

73. The method of any one of claims 62-69, wherein the subject is administered the therapeutically effective amount of the compound within 15 minutes before or after ingesting the food.

74. The method of any one of claims 62-69, wherein the subject is administered the therapeutically effective amount of the compound within 5 minutes before or after ingesting the food.

75. The method of any one of claims 62-75, wherein the food comprises a high fat meal.

76. The method of any one of claims 62-75, wherein the food comprises a regular meal.

77. The method of any one of claims 62-75, wherein the food comprises at least 50 calories.

78. The method of any one of claims 62-75, wherein the food comprises at least 100 calories.

79. The method of any one of claims 62-75, wherein the food comprises at least 200 calories.

80. The method of any one of claims 62-75, wherein the food comprises at least 300 calories.

81. The method of any one of claim 62-80, wherein administering the therapeutically effective amount of the compound substantially contemporaneously with food increases the bioavailability of the compound compared to administration without food, wherein bioavailability is based on a comparison of AUC values.

82. The method of claim 81, wherein the bioavailability is increased by about 10% or greater.

83. The method of claim 81, wherein the bioavailability is increased by about 20% or greater.

84. The method of claim 81, wherein the bioavailability is increased by about 30% or greater.

85. The method of claim 81, wherein the bioavailability is increased by about 40% or greater.

86. The method of claim 81, wherein the bioavailability is increased by about 50% or greater.

87. The method of claim 81, wherein the bioavailability is increased by about 55% or greater.

88. A method of treating a CNS-related disorder in a subject, the method comprising administering to the subject a therapeutically effective amount of a compound substantially contemporaneously with food, the compound having the formula: 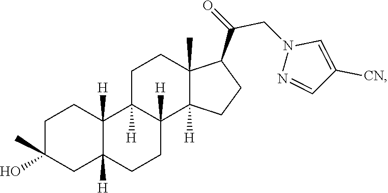
wherein the administration results in an increase in the bioavailability of the compound compared to administration without food, wherein bioavailability is based on a comparison of AUC values.

89. The method of claim 88, wherein the therapeutically effective amount is in a solid dosage form.

90. The method of claim 89, wherein the solid dosage form is a tablet or a capsule.

91. The method of any one of claims 87-89, wherein the bioavailability is increased by about 10% or greater.

92. The method of any one of claims 87-89, wherein the bioavailability is increased by about 20% or greater.

93. The method of any one of claims 87-89, wherein the bioavailability is increased by about 30% or greater.

94. The method of any one of claims 87-89, wherein the bioavailability is increased by about 40% or greater.

95. The method of any one of claims 87-89, wherein the bioavailability is increased by about 50% or greater.

96. The method of any one of claims 87-89, wherein the bioavailability is increased by about 55% or greater.

97. The method of any one of claims 87-96, wherein the AUC value is AUC₀₋ₜ, wherein AUC₀₋ₜ is the AUC from the time of dosing to the last quantifiable concentration.

98. The method of any one of claims 87-97, wherein the CNS-related disorder is a GABA related disease or disorder.

99. The method of any one of claims 87-97, wherein the CNS-related disorder is a mood disorder.

100. The method of any one of claims 87-97, wherein the CNS-related disorder is a movement disorder.

101. The method of any one of claims 87-97, wherein the CNS-related disorder is postpartum depression.

102. The method of any one of claims 87-97, wherein the CNS-related disorder is major depressive disorder.

103. The method of any one of claims 87-97, wherein the CNS-related disorder is essential tremor.

104. The method of any one of claims 87-97, wherein the movement disorder is Parkinson's disease.

105. The method of claim 102, wherein the baseline HAM-D total score of the subject is at least 24.

106. The method of claim 102, wherein the baseline HAM-D total score of the subject is at least 18.

107. The method of claim 102, wherein the baseline HAM-D total score of the subject is at between and including 19 and 22.

108. The method of claim 102, wherein the baseline HAM-D total score of the subject is at between and including 14 and 18.

109. The method of claim 102, wherein the baseline HAM-D total score of the subject is greater than or equal to 23.

110. The method of claim 102, wherein the compound is administered to the subject once a day for at least 14 days.

111. The method of any one of claims 105-110, wherein the HAM-D total score associated with treating the subject with the compound is no more than a number ranging from 6 to 8.

112. The method of any one of claims 105-111, wherein the HAM-D total score associated with treating the subject with the compound is no more than 7.

113. The method of any one of claims 105-112, wherein the decrease in HAM-D total score after treating the subject with Compound 1 relative to the baseline HAM-D total score is at least 15.

114. The method of any one of claims 105-113, wherein the decrease in HAM-D total score after treating the subject with Compound 1 relative to the baseline HAM-D total score is at least 17.

115. The method of any one of claims 105-114, wherein the subject is administered the compound at night.

116. The method of any one of claims 105-115, wherein the subject is administered the compound no longer than 1 hour before the subject sleeps.

117. The method of any one of claims 105-116, wherein the subject is administered the compound no longer than 15 minutes before the subject sleeps.

118. The method of any one of claims 105-117, wherein the compound is administered chronically.

119. The method of any one of claim 1-49 or 88-118, wherein Compound 1 exists as Form C.

120. The article of manufacture of any one of claims 50-61, wherein Compound 1 exists as Form C.
